(19) United States
(12) Patent Application Publication
(10) Pub. No.: US 2019/0209858 A1
(43) Pub. Date: Jul. 11, 2019

(54) CIRCADIAN STIMULUS VIA IMAGE PROCESSING OR ALGORITHM

(71) Applicant: ABL IP HOLDING LLC, Conyers, GA (US)

(72) Inventors: Christopher D. Slaughter, Littleton, CO (US); Daniel Aaron Weiss, Tucker, GA (US)

(21) Appl. No.: 15/867,145

(22) Filed: Jan. 10, 2018

(51) Int. Cl. A61N 5/06; A61B 3/00; G06F 17/30

(52) U.S. Cl. CPC A61N 5/0613; A61B 3/0008; A61N 2005/0652; A61N 2005/0635; A61N 2005/0663; G06F 17/30247

(57) ABSTRACT

Disclosed is an example of a system for selectively influencing the circadian system of a human being, or other human stimulus factor, while simultaneously making minimal shifts to a specified operational parameter of the light source, such as correlated color temperature (CCT), color rendering index, look on a fixture, net lumen output, or a power consumption requirement. An example involves image processing to produce drive signals for emitters of a source array in a luminaire and an associated illumination control function to continuously meet a specific melanopic lux or circadian stimulus value, while adjusting/optimizing one or more other operational parameters of the source emitter outputs to achieve or maintain the operational parameter, such as light output level or power consumption, to within some acceptable degree of tolerance.

### TECHNICAL FIELD

The present subject matter relates to techniques and equipment to vary intensity or color characteristic or spectral power distribution of light emitted by a light emitter of an array to promote a desired human stimulus impact (e.g. circadian stimulus, circadian impact, melanopic lux, alerting sensitivity of the human visual system, or the like) while controlling the array of emitters to achieve or maintain a light output at least within a tolerance of a predetermined level of a desired parameter of combined emitted light, e.g. for general illumination purposes.

### BACKGROUND

Electrical lighting has become commonplace in modern society. Electrical lighting devices are commonly deployed, for example, in homes and buildings of commercial and other enterprise establishments. Traditional general lighting devices have tended to be relatively dumb, in that they can be turned ON and OFF, and in some cases may be dimmed, usually in response to user activation of a relatively simple input device. Such lighting devices have also been controlled in response to ambient light detectors that turn on a light only when ambient light is at or below a threshold (e.g. as the sun goes down) and in response to occupancy sensors (e.g. to turn ON light when a room is occupied and to turn the light OFF when the room is no longer occupied for some period). Often such devices are controlled individually or as relatively small groups at separate locations. Traditional control algorithms involved setting an operational parameter of the light output, such as intensity and/or color or other spectral characteristic, and then maintaining the set condition within some minimal variance for a relatively long period of time, e.g. over a work day or over a period occupancy. Often, the setting(s) would apply to most if not all sources emitting light into a particular illuminated space, for example, so that the illumination throughout the space would have a relatively uniform characteristic.

It has been recognized, however, that variation in lighting characteristics and/or variations over time may have desirable effects on occupants. Simulation of natural lighting, for example, may enhance performance of workers occupying the illuminated space. Other variations may produce adverse effects desired by an operator of the lighting device or system, for example, to encourage people not to linger too long in a particular area. There have been proposals and/or product offerings involving use of displays as lighting devices mounted on ceilings or walls, where the lighting device displays are driven by still image or video signals. In some cases, outside cameras capture video of outside conditions, and the lighting devices display the videos to provide indoor illumination.

Current lighting fixtures may be configured to affect the circadian system through the photosensitive retinal ganglion cells, which have a sensitivity curve which is measured by melanopic lux. There are additional metrics currently being developed by the Lighting Research Center called Circadian Stimulus and others. In general, the melanopic lux sensitivity curve peaks somewhere around the 480 nm wavelength (blueish), and melanopic lux attempts to put numerical values to the amount of stimulus which suppresses melatonin and helps to regulate an internal circadian biological clock. This numeric value for optical human stimulus is useful and utilized by requirements such as the WELL Building Standard. Recommendations for this circadian stimulus have a numerical dosage level (amount and duration), and time of day components. Currently the major industry methods to address this recommended numeric value for stimulus involve either (1) dimming up or down a single color temperature source of a particular white light (such as 4000K LEDs) or (2) using a tunable-white style light fixture which has multiple LEDs of different colors/characteristics for variable control of correlated color temperature (CCT) adjustment that can also dim up/down. To blend one of these approaches with net lumen output for minimum/maximum illuminance levels in a space to meet specific circadian stimulus targets can be challenging. In addition there are more efficacious ways of providing circadian stimulus, such as utilizing a blue LED which is in the peak of the sensitivity curve.

Hence, there is room for further improvement in technologies to provide effective optical stimulus and provide suitable levels and/or qualities of light for general illumination.

### SUMMARY

The technology examples described herein address one or more of the needs outlined above by selectively generating light to influence the circadian system of a human being, or other human stimulus factor, while simultaneously making minimal shifts to a specified parameter of light emission operation the light source. Examples may use a variety of processing techniques, such as image processing or random or other algorithmic processing, to achieve a desired human stimulus impact without an unduly adverse impact on a desired parameter related to artificial illumination provided by the lighting device.

A lighting device, for example, includes a luminaire and a controller. The luminaire has a light source that includes independently controllable light emitters configured to emit light. Each respective light emitter is configured to be controlled to vary intensity or a color characteristic of light emitted from the respective light emitter. The individually controllable light emitters together are configured to emit light in a manner meeting a predetermined level of an operational parameter of the light source. The controller is coupled to control the light emitters of the source in the luminaire. The controller obtains a setting corresponding to a desired human stimulus impact to be provided by the luminaire. Based on the setting, the control controls at least one of the light emitters to vary intensity or color characteristic or spectral power distribution of light emitted by the at least one light emitter, to promote the desired human stimulus impact. During the variation of the intensity or color characteristic or spectral power distribution of light emitted by the at least one light emitter, operation of the light source also is controlled to cause the emitters of the source to maintain operation at least within a tolerance of the predetermined level of the parameter of operation the light source.

In another example, a lighting device includes a luminaire having a light source. The light source includes an array of controllable light emitters configured to generate light output representing an image. Each respective light emitter is configured to be controlled to vary intensity and a color characteristic of light emitted from the respective light emitter. This example lighting device also includes a processor coupled to control the light emitters and a memory coupled to the processor that stores data of each of a number of images. The processor is configured to obtain a setting corresponding to a desired human stimulus impact to be promoted by light emitted by the controllable light emitters, and a value specifying a predetermined level of a parameter of light source operation. An image is selected that should promote the desired human stimulus impact. Based on the data of the selected image, the processor controls the light emitters to generate light output representing the selected image. Concurrently, the processor controls the light source to cause the emitters of the source to maintain operation at least within a tolerance of the predetermined level of the parameter of operation the light source.

Additional objects, advantages and novel features of the examples will be set forth in part in the description which follows, and in part will become apparent to those skilled in the art upon examination of the following and the accompanying drawings or may be learned by production or operation of the examples. The objects and advantages of the present subject matter may be realized and attained by means of the methodologies, instrumentalities and combinations particularly pointed out in the appended claims.

### BRIEF DESCRIPTION OF THE DRAWINGS

The drawing figures depict one or more implementations, by way of example only, not by way of limitations. In the figures, like reference numerals refer to the same or similar elements.

FIG. 1 is a simplified flow chart of an example of a process for coordinated control of an operational parameter and a variable lighting characteristic intended to promote a desired human stimulus impact.

FIG. 1A graphically depicts a simple example of variation in a stimulus impact setting and variation of an operational parameter, relative to a time schedule.

FIG. 2 is a graph of two examples of human light sensitivity functions.

FIG. 2A is a graph of several different spectral power distributions substantially achieving the same coordinated color characteristic.

FIG. 3 is a graph of X, Y and Z tristimulus functions.

FIG. 4 is a graph of relative melanopic and photopic functions.

FIG. 5 is a graph depicting normalized photopic sensitivity.

FIG. 6 is a graph of a sample spectral power distribution (SPD) curve.

FIG. 7 illustrates a calculated sensitivity curve for melanopsin.

FIG. 8 is a graph of the spectral power distributions of individual LEDs as may be used as pixel emitters an example light source array.

FIG. 8A is a bar graph useful in discussion of an example of overall adjustments of spectral outputs from different color emitters of a light source.

FIG. 9 is a high level functional block diagram of a lighting device that includes a luminaire and a controller.

FIGS. 10A to 10C are plan views of several example configurations of luminaires and light source emitters arrays, with different enlarged examples of pixel emitter configurations.

FIG. 11 is a high-level functional block diagram of a system including a number lighting devices similar to that of FIG. 9, which that may communicate with other system elements at a premises and/or with external computers via a wide area network.

FIGS. 12A and 12B are a sample image and a spectrum distribution graph respectively, for a color image producing light of a low melanopic lux value.

FIGS. 13A and 13B are a modified version of the sample image and a corresponding modified spectrum distribution graph respectively, for a blue-weighted image.

FIGS. 14A to 14D are plan views of a luminaire output of a portion of an image, where the image data has been processed to provide four different values of melanopic lux.

FIG. 15 depicts an image processing flow in the form of three progressive states of an image, representing light output states to promote different degrees of human stimulus impact.

FIGS. 16A to 16D depict by way of example several images of different characteristics that may be selected for output to promote different degrees of human stimulus impact.

FIGS. 17A and 17B are a flow chart and associated normalized CCT distribution graph, respectively for a two-channel white tuning example.

FIGS. 18A and 18B depict two different states of a luminaire output providing the same coordinated color temperature (CCT) values for white light output, but which offer different visual effects, and may be selected by a pseudo-random processing algorithm.

FIG. 19 is a simplified flow chart example of a general work flow for algorithmically determining intensity and spectral characteristics for the image pixels of the light output.

FIG. 20 is a graph of normalized spectrum distributions for red (R), green (G), blue, (B) and white (W) LED type light emitters, useful in understanding the color gamut achievable with a light source array using such emitters.

FIGS. 21A to 26B are gamut plots and graphs regarding chromaticity, for different lighting operational parameters.

FIG. 27 is a flow chart of a process for factor optimization.

FIG. 28 graphically illustrates several lighting parameters and related stimulus impact values, for several different outcomes of the factor optimization process.

FIG. 29 is a simplified functional block diagram of a computer that may be configured as a host or server, which may communicate with a lighting device.

FIG. 30 is a simplified functional block diagram of a personal computer or other similar user terminal device, which may communicate with a lighting device.

FIG. 31 is a simplified functional block diagram of a mobile device, as an alternate example of a user terminal device, for possible communication with a lighting device.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and/or circuitry have been described at a relatively high-level, without detail, in order to avoid unnecessarily obscuring aspects of the present teachings.

The various examples disclosed herein relate to lighting equipment and techniques to selectively generate light to influence a human stimulus factor. In some examples, while providing light of a characteristic to promote a particular stimulus impact, the light source also is concurrently controlled to achieve or maintain an operational parameter of the source during light output at least within a tolerance of a predetermined level of a target value for the operational parameter. A typical lighting device includes a luminaire with a suitable controllable light source and a controller. The controller may be integrated in or located fairly close to the luminaire. However, some of the control related functions may be implemented on a more distributed basis, e.g. with high intensity image processing for some of the examples performed “in the cloud” so to speak.

At a high level, a stimulus metric setting, such as a value of melanopic lux, varies over a period of time in a manner to promote variable stimulus impact. A lighting device operates in response to variations in the stimulus metric setting to produce corresponding changes in light output from a source included as part of the lighting device. In most examples, the source includes an array of multi-channel controllable emitters together capable of outputting light of an image. Aspects of the image light output are varied in response to the variations in the stimulus metric setting.

The examples below may also adjust other aspects of the image light output to compensate for the changes due to the variations in the stimulus metric setting, to optimize operation of the relative to one or more operational parameters, such as power consumption, overall light output intensity (e.g. total flux in lumens), color rendition (e.g. CRI or TM-30-15), or overall spectral characteristic (e.g. CCT, R9, etc.). Optimization may fairly precisely maintain the operational parameter but will at least keep the operational parameter within some tolerance of a target value, e.g. within ±10%, ±5%, ±2%, ±1% or ±0.5% of a respective target.

The term “luminaire,” as used herein, is intended to encompass essentially any type of device that processes energy to generate or supply artificial light, for example, for general illumination of a space intended for occupancy or observation, typically by a living organism typically a human that can take advantage of or be affected in some desired manner by the light emitted from the device. However, a luminaire may provide light for use by automated equipment, such as sensors/monitors, robots, etc. that may occupy or observe the illuminated space, instead of or in addition to light provided for an organism. However, it is also possible that one or more luminaires in or on a particular premises have other lighting purposes, such as signage for an entrance or to indicate an exit. In most examples, the luminaire(s) illuminate a space or area of a premises to a level useful for a human in or passing through the space, e.g. general illumination of a room or corridor in a building or of an outdoor space such as a street, sidewalk, parking lot or performance venue. The actual source of illumination light in or supplying the light for a luminaire may be any type of artificial light emitting device having suitable individually controllable operation characteristics, several examples of which are included in the discussions below.

The illumination light output of a luminaire, for example, may have an intensity and/or other characteristic(s) that satisfy an industry acceptable performance standard for a general lighting application. The performance standard may vary for different uses or applications of the illuminated space, for example, as between residential, office, manufacturing, warehouse, or retail spaces. For selectively influencing a human stimulus factor, parameters of the light output of the luminaire also are variable to provide desired stimulus impacts. As discussed in more detail later, the control functions enable variation of light to promote desired stimulus impacts yet achieve and maintain a parameter of illumination light output within a suitable tolerance (e.g. within ±10%) of a parameter such as industry acceptable intensity or color characteristic or spectral power distribution for the overall lighting device or luminaire light output.

The term “color characteristic” may refer to any number of color metrics, examples of which include intensity to convey hue, saturation, color rendition, or in certain color spaces light intensity. For instance, a chromaticity coordinate (x,y) derived from the 1931 CIE chromaticity diagram (or subsequent color coordinate spaces) would be one example of a color characteristic. Other examples would be XYZ tristimulus values, CCT (correlated color temperature); or CRI (color rendering index), CQS (color quality scale) or TM-30 for color rendition.

The term “spectral power distribution” (SPD) refers to a net representation of the radiometric power (usually watts) emitted by a light source (which could be comprised of multiple light sources) at each wavelength (usually nm) and generally graphically depicted in the range of the visible region of the electromagnetic spectrum.

Terms such as “artificial lighting,” as used herein, are intended to encompass essentially any type of lighting that a device produces light by processing of electrical power to generate the light. An artificial lighting device, for example, may take the form of a lamp, light fixture, or other luminaire that incorporates a suitable light source, where the light source by itself contains no intelligence or communication capability, such as one or more LEDs or the like, or a lamp (e.g. “regular light bulbs”) of any suitable type.

In several illustrated examples, such a luminaire may take the form of a light fixture, such as a pendant or drop light or a downlight, or wall wash light or the like. Other fixture type luminaire mounting arrangements are possible. For example, at least some implementations of the luminaire may be surface mounted on or recess mounted in a wall, ceiling or floor. Orientation of the example luminaires and components thereof are shown in some of the drawings and described below by way of non-limiting examples only. The luminaire with the lighting component(s) may take other forms, such as lamps (e.g. table or floor lamps or street lamps) or the like. Additional devices, such as fixed or controllable optical elements, may be included in the luminaire, e.g. to distribute light output from the light source in a particular manner.

Terms such as “lighting device” or “lighting apparatus,” as used herein, are intended to encompass essentially any combination of an example of a luminaire discussed herein with other elements such as electronics and/or support structure, to operate and/or install the particular luminaire implementation. Such electronics hardware, for example, may include some or all of the appropriate driver(s) for the illumination light source, any associated control processor or alternative higher level control circuitry, and/or data communication interface(s). The electronics for driving and/or controlling the lighting component(s) may be incorporated within the luminaire or located separately and coupled by appropriate means to the light source component(s) of the luminaire.

The term “lighting system,” as used herein, is intended to encompass essentially any type of system that either includes a number of such lighting devices coupled together for data communication or a lighting device coupled together for data communication with one or more control devices, such as wall switches, control panels, remote controls, central lighting or building control systems, servers, cloud services, etc.

In several of the examples, the lighting device is configurable by programming instructions and/or setting data, e.g. which may be communicated to a processor of the lighting device via a data communication network of a lighting system. Configurable aspects of lighting device operation may include one or more of: data of a selected image (still or video) configured to cause lighting components of the luminaire to output light of the image so as to promote a desired human stimulus impact, a modified or processed image (still or video) configured to cause lighting components of the luminaire to output light of the image so as to promote a desired human stimulus impact, any parameter for an algorithm that controls the lighting components of the luminaire to output light so as to promote a desired human stimulus impact, programming or data for such an algorithm or an update thereof, any target parameter setting for one or more operational parameters of light source operation, and any parameter setting for one or more characteristics of light output intended to promote desired human stimulus impact(s).

The term “coupled” as used herein refers to any logical, physical or electrical connection, link or the like by which signals produced by one element are imparted to another “coupled” element. Unless described otherwise, coupled components, elements or devices are not necessarily directly connected to one another and may be separated by intermediate components, elements, devices or communication media that may modify, manipulate or carry the signals.

Light output from the luminaire may carry information, such as a code (e.g. to identify the luminaire or its location) or downstream transmission of communication signaling and/or user data. The light based data transmission may involve modulation or otherwise adjusting parameters (e.g. intensity, color characteristic, flicker or light output distribution) of the illumination light output.

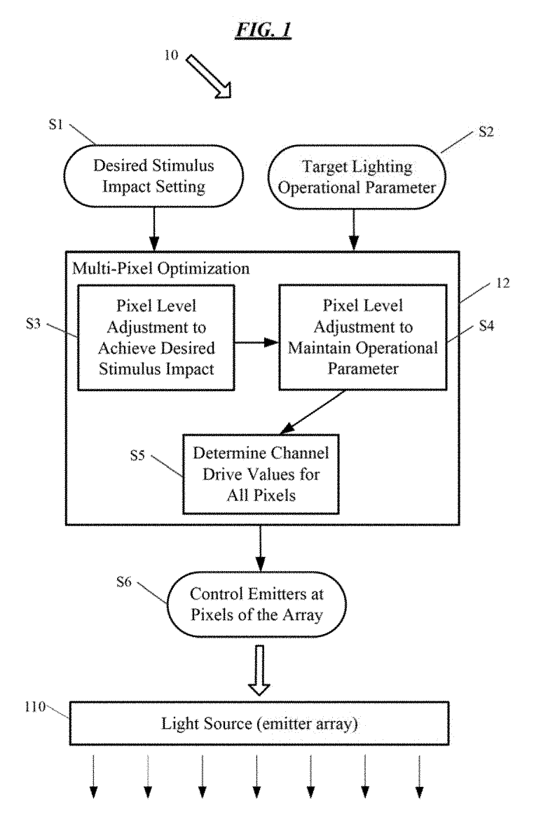

Reference now is made in detail to the examples illustrated in the accompanying drawings and discussed below. FIG. 1 illustrates a process 10 for coordinated control of an operational parameter of an illumination light source and a variable lighting characteristic of the source output intended to promote a desired human stimulus impact.

At a high level, such a procedure 10 involves obtaining a setting (step S1) corresponding to a desired human or other biological stimulus impact to be provided by the light emitted by the plurality of controllable light emitters. The present approach may be used for a variety of types of human stimulus factors involving light and may be measured by various metrics, such as melanopic lux, circadian stimulus, circadian impact, alerting sensitivity of the human visual system, or the like. The procedure may also use or process a target parameter of lighting operation, as obtained by the process at S2. Examples of operational parameters include an overall intensity desired for illumination of a particular space at luminaire output or at task level, overall power consumption, various measures of overall spectral or color characteristic(s) of the light output or at task level, etc. The stimulus impact setting (e.g. a value of melanopic lux or circadian lux) and operational parameter value are inputs to a multi-pixel optimization routine 12, which implements a number of steps shown in the simple flow-chart example of FIG. 1.

The procedure 10 of FIG. 1 results in control of a multi emitter array type light source 110. Although not separately shown, initial pixel level output values to achieve the target light performance identified by the parameter value indicated in step S2 may be retrieved or determined by appropriate processing. The multi-pixel optimization routine 12 determines new pixel level output values responsive to the desired stimulus impact setting and optimized for the target lighting operational parameter. The optimization, for example, may result in modification of the pixel outputs in a non-uniform way.

More specifically, in the example, the multi-pixel optimization routine 12 implements a step S3 to determine one or more pixel level adjustments to achieve the desired stimulus impact indicated by the setting value obtained in step S1. Step S3 will typically involve a modification of the initial pixel level operational values in a non-uniform way for one or more emitters of the source 110 so as to promote the desired stimulus impact. The multi-pixel optimization routine implements a step S4 to optimize light source performance, to keep the overall emitted light and/or other aspect (e.g. power) of the light source within a tolerance (e.g. ±10%) of the target value for the light output. Step S4 will involve determining adjustments to pixel level values for lighting outputs of one or more controllable light emitters, typically including some adjustment (which may be non-uniform) of some number of other emitters of the of the array of the light source 110.

The results of step S4 are processed (S5) to determine actual drive values for the channels of each of the controllable emitters of the array of the light source 110. As discussed later, an appropriate driver circuit responds to these values to drive the actual emitters of the particular implementation of the light source array. In this way, based on the stimulus impact setting, at least one of the light emitters of the source 110 is controlled to vary intensity or color characteristic or spectral power distribution of light emitted by the at least one light emitter to promote the desired human stimulus impact. Also, during the variation of the intensity or color characteristic or spectral power distribution of light emitted by the at least one light emitter, operation of at least one light emitter of the array is controlled to maintain operation of the light source 110 at least within the tolerance of the predetermined level of the target for the overall light source operational parameter. Viewing the output of the luminaire from the illuminated space, the output may appear non-uniform, for example, with different regions having visibly different colors or intensities.

For many applications of the processing technique, the intent is to vary the stimulus impact, for example, over some period of time of operation of the luminaire 110, such as over the daytime hours of typical operation of an office or similar enterprise installation. In such cases, although the operational parameter value obtained at S2 may stay the same, the desired impact stimulus setting obtained at S1 will change, and other steps S3 to S6 may be repeated based on the new impact stimulus setting to change the light output from the light source 110.

It may be helpful to consider an example. FIG. 1A that graphically depicts variation in a stimulus impact setting and variation to generally maintain an operational parameter, relative to a time schedule. For purposes the example, the stimulus metric is melanopic lux (ML), units of the setting value for which are shown on the left vertical axis in the drawing. In this example, the operational parameter that is to be maintained is electrical power consumption by the light source 110, as measured in Watts (W) indicated along the right vertical axis. The horizontal axis represents time of day.

The solid line graph in the drawing represents scheduled stepped changes in the value of the setting for melanopic lux over time of day; and for discussion purposes, it is assumed that the luminaire is ON continuously throughout the illustrated period of the day. The procedure of FIG. 1 will operate the light source 110 to output light of an image that closely tracks the solid line graph. For example, early in the morning, per the setting, the light source 110 will output light at about 150 melanopic lux until 8:30 am. At around 8:30 am, the controller that controls operation of the light source 110 will respond to the time of day and adjust the operation of the of the light source 110 to a new setting, in the example, to cause the light source 110 to output light at approximately 350 melanopic lux. In a similar fashion, at about 9:45 am, the controller associated with the light source 110 will respond to the time of day and adjust the operation of the of the light source 110 to a new setting, in the example, to cause the light source 110 to output light at approximately 575 melanopic lux. The light output will stay at this level of melanopic lux into the afternoon.

Continuing with the example of FIG. 1A, at about 2:15 pm, the controller associated with the light source 110 will respond to the time of day and adjust the operation of the of the light source 110 to a new setting, in the example, to cause the light source 110 to again output light at approximately 350 melanopic lux. In a similar fashion, at about 3:30 pm, the controller will respond to the time of day and adjust the operation of the of the light source 110 to a new setting, in the example, to cause the light source 110 to output light at approximately 150 melanopic lux for the remainder of the daily operation of the luminaire.

During these variations in luminaire operation to provide specified settings for the melanopic lux of the output light, the controller that is operating the light source 110 is adjusting the operations of the emitters at the pixel of the array of the source to also maintain the operational parameter at or near the specified target value. In the example of FIG. 1A, the parameter is power consumption; and for discussion purposes, it is assumed that the target value is 1.0 W (at the level corresponding to the dot-dash line in the drawing). The control programming may configure the controller associated with the light source 110 to keep the power consumption in the range from 0.95 W to 1.05 W (±5% of the 1 W target value). In actual operation, the controller associated with the light source 110 may be able to keep power consumption even closer to the target parameter value. The line of small-dots is a representative example in which the power consumed by the light source 110 varies in the range from 0.98 W to 1.02 W (±2% of the 1 W target value).

A time schedule is used as the example of a basis to change the desired stimulus impact setting. The setting change, however, may be based on other factors, such as sensed intensity and/or spectral characteristic of ambient light, sensed temperature in the illuminated space, sensed number of occupants, received user inputs, or the like. Also, for ease of illustration and discussion, the example of FIG. 1A included stepped changes over time. Other implementations may provide more frequent changes, different levels or degrees of changes and/or continuous change as a function of time.

In the examples below, a luminaire includes at least one or more components, typically an array of individually controllable emitters, forming a lighting source 110 for generating the artificial illumination light for a general lighting application. The array of emitters also may be controllable to produce and output light in a manner representing a stationary or moving image. At a distance from the luminaire, the light output from the luminaire provides artificial illumination and the displayed image may be visible to an observer in the space lighted by the luminaire.

Traditionally, tuning the quality of white light in illumination, e.g. to change color temperature (CCT) or the like, involves varying the quality in a particular manner across the entire output area of the luminaire. The lighting device here, for example, may be configured to vary the CCT or the like across the device output, much like an image display, yet coordinate the variations across the output in a manner that the combined light (e.g. at a distance from the output) maintains a desired CCT value or the like, within a suitable degree of tolerance.

Emitters at points of the array also are individually controllable, for example, to vary one or more intensity, color characteristic or spectral power distribution of light emitted by the light emitter at each pixel point of the array. Although other types of sources may be used, the examples utilize LED based light sources, with emitters at points of an array for emission of light of pixels of an image. Each such emitter has two or more LEDs of each of several different spectral characteristics, e.g. different colors (e.g. RGB) or different color temperatures of white or combinations thereof (e.g. RGBW, or RGBWA). The LEDs are controlled to affect the circadian system or other human stimulus system through the photosensitive retinal ganglion cells. The human retinal ganglion cells have a sensitivity curve which with a sensitivity curve peak somewhere around the 480 nm wavelength (blueish). The intent is to optimize outputs of various LEDs of the array of the source 110 to both promote the desired stimulus impact and to maintain adequate performance of the lighting device. Several specific techniques to determine suitable drive channel values for the array, such as image selection, image processing, and white tuning will be discussed in greater detail later. It may be helpful first to consider the underlying theory of operation.

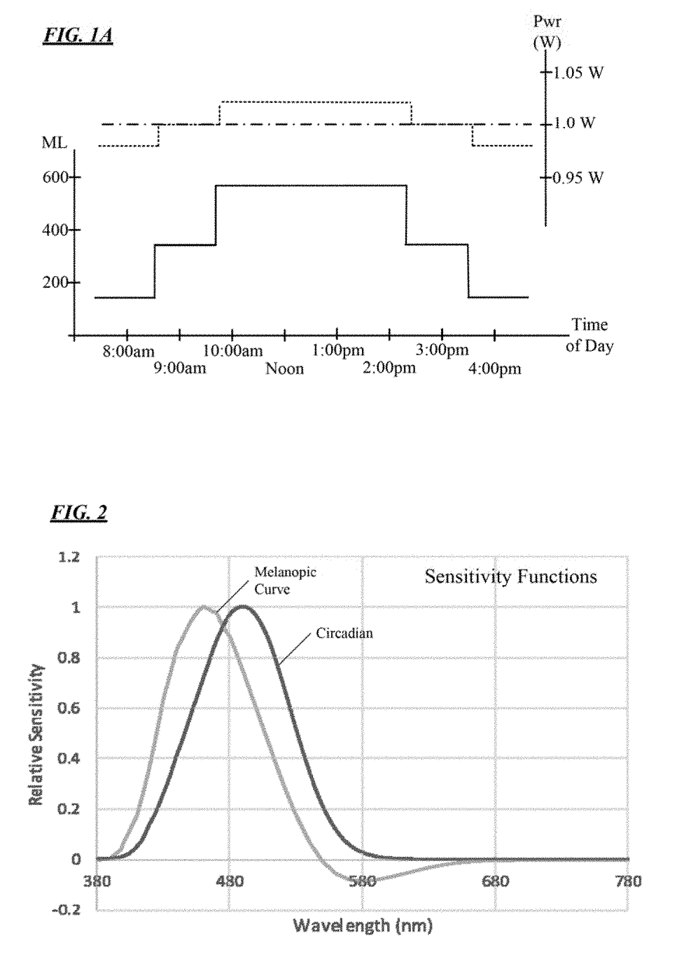

The graph of FIG. 2 illustrates two human sensitivity curves, that is to say relative human retinal sensitivity as a function of wavelength. One curve is the circadian sensitivity curve, which represents the general impact or stimulus on a circadian rhythm of the human body in response to various visible wavelengths of light. The melanopic curve represent the relative sensitivity of a human receiving light via the retina, with respect to visible wavelengths in terms of responsive suppression of the body's production of potentially sleep promoting melatonin.

As shown by the curves in FIG. 2, there tend to be sensitivity curve peaks somewhere around the 480 nm wavelength (blueish). The parameter melanopic lux is a scientific attempt to put numerical values to the amount of stimulus which suppresses melatonin and helps to regulate an internal circadian biological clock. Recommendations for this circadian stimulus have a numerical dosage level (amount and duration) as well as time of day components.

Using prior luminaire systems, the ability to influence the biological system effects other metrics of lighting device operation such as power, color rendition, or visual aesthetics, in a negative way. It is difficult for designers to maintain the intent of their design while also stimulating (or negatively stimulating) the biological systems of humans or other biological creatures.

The examples described below improve over the prior luminaire technologies by combined processing to implement the variation to promote the desired human stimulus impact based on the setting in a manner coordinated with adjustment of other aspects of luminaire performance so as to minimize the negative impact on the lighting performance of the luminaire, for example, to balance the setting for stimulus impact (e.g. a value of melanopic lux or circadian lux) with respect to one or more other aspects of luminaire of lighting device performance, such as net power usage, overall CRI, net light output, overall color rendition, etc.

At a high level, the examples address this optimization or balancing by determining outputs for multiple controllable channels of emitters of the array of a light source 110. These outputs may be determined so as to always meet a specific melanopic lux or circadian stimulus value and for example adjust for light output or power requirements as an overall optimization parameter. There is an initial bounding metric of numerical circadian stimulus or melanopic lux (generically known as Circadian Impact), and then an overall optimization parameter such as net CCT, x,y coordinate, net power usage, CRI, TM-30-15, R9 or net light output.

For example, with the luminaire fixture with pixel-level control, data of an image or representational image (e.g. blurred and abstract) can be produced from a file of image data or from an algorithm, for optimized light output from the array of the light source 110. The data of the image may be adjusted for example to maintain the macro look of the luminaire output but modified in net spectrum of each pixel output to keep a non-uniform difference between the pixels defined by an algorithm to balance the variables described above. The aggregate output (the flux) of the entire set of pixels combines to produce the desired output. For example, three pixels instead of all being on the black body diagram at 4000K CCT and delivering a specific melanopic lux, could be spread out in a triangle on a chromaticity diagram and deliver the same melanopic lux.

Lighting device examples that utilize LED based light sources, with emitters at points of an array for emission of light of pixels of an image, may utilize image processing of the LED drive signals and an associated algorithm, to continuously meet a varying melanopic lux or circadian stimulus value, while adjusting/optimizing one or more other operational parameters of the LED light fixture, such as light output or power. For instance, on a 2 channel white tuning solution, power usage or CCT can be optimized while other variables are dependent upon the first. However on a 3 channel white tuning solution: CCT, or lumen output can be optimized and then power becomes dependent. When a fourth channel (such as a blue chip) is added, it adds another dimension to the ability to optimize parameters.

In a simple example, promotion of a desired stimulus impact may involve an increase in blue light emission from the source 110, at a particular time or period of time during the day, to increase suppression of melatonin in humans occupying a particular enterprise space. Depending on the amount of the increase in blue light output, a number of pixel point emitters of the array of the source 110 are selected and the outputs thereof are adjusted to increase blue light output. As noted, the emitters at the points of the array are multi-channel (multi-color or multiple spectral characteristic emitters). For discussion of this example, we will assume that the emitter at each point or pixel of the array is an RGB type emitter. Hence, an increase in blue output of the selected RGB emitters may or may not coincide with a decrease in red or green emissions of those pixel-point emitters. Some aspects of source operation, however, are adjusted to compensate for the operational effects of the adjustment in blue emissions.

For example, if the intent is to maintain overall light output intensity of the light, then some number of other emitters at points of the array that were emitting non-blue light (e.g. red or green light or combinations thereof) are turned down to emit less light collectively by an amount to approximately offset the increase in overall intensity that otherwise would result from the increase in blue emissions. If intensity is the only target parameter being maintained, it may be possible to very nearly achieve and maintain the target intensity while outputting light from the source 110 with the increased blue emissions.

As another example, if the intent is to maintain overall power consumption by the operation of the light source 110, then the drive currents supplied to some number the emitters at points of the array that were emitting non-blue light (e.g. red or green light or combinations thereof) are turned down to offset the increase in power consumption by blue emitters. If overall power consumption by the source 110 is the only target parameter being maintained, it may be possible to very nearly achieve and maintain the target power consumption level while outputting light from the source 110 with the increased blue emissions.

If the intent is to optimize two or more parameters of the overall operations of the light source while changing the light characteristic promoting the stimulus impact, the solution becomes more complex. In many cases, however, the optimization solution still is able to maintain a number of such parameters within respective tolerances of the applicable parameter target values. Continuing with the blue light increase example, consider next a case in which the intent is to maintain both overall intensity and total power consumption within tolerances of target values for intensity and power. The actual diodes for red, green and blue forming the emitters at the point of the array may not have the same efficiency, e.g. different diode types may consume different amounts of power to achieve similar light output levels. Hence, an optimization solution might permit up to a 2% increase in power consumption and up to a 5% drop in overall intensity coordinated with the increase in blue light emission to increase melanopic lux based on a desired stimulus impact setting.

The controller may control a color characteristic at the emitter level and/or may optimize an overall color characteristic of the luminaire output. As noted, based on a setting, the control controls at least one of the light emitters to vary intensity or color characteristic or spectral power distribution of light emitted by the at least one light emitter, to promote the desired human stimulus impact. During this controlled variation at one or more of the light emitters, operation of the light source also is controlled to cause the emitters of the source to maintain operation at least within a tolerance of the predetermined level of the parameter of operation the light source, and that parameter of overall operation may be a color characteristic, power consumption, overall light output intensity, or the like.

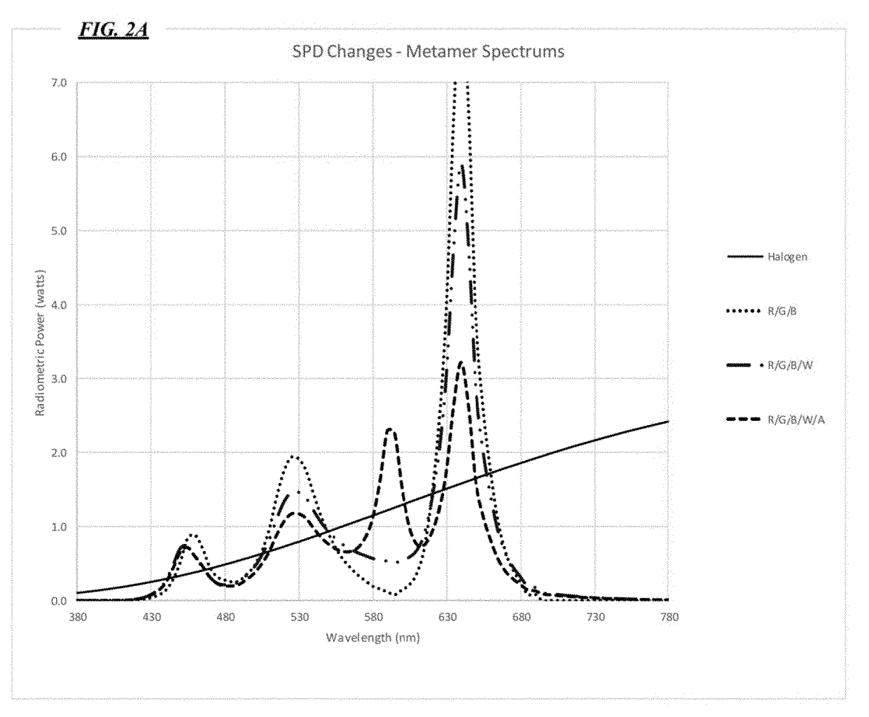

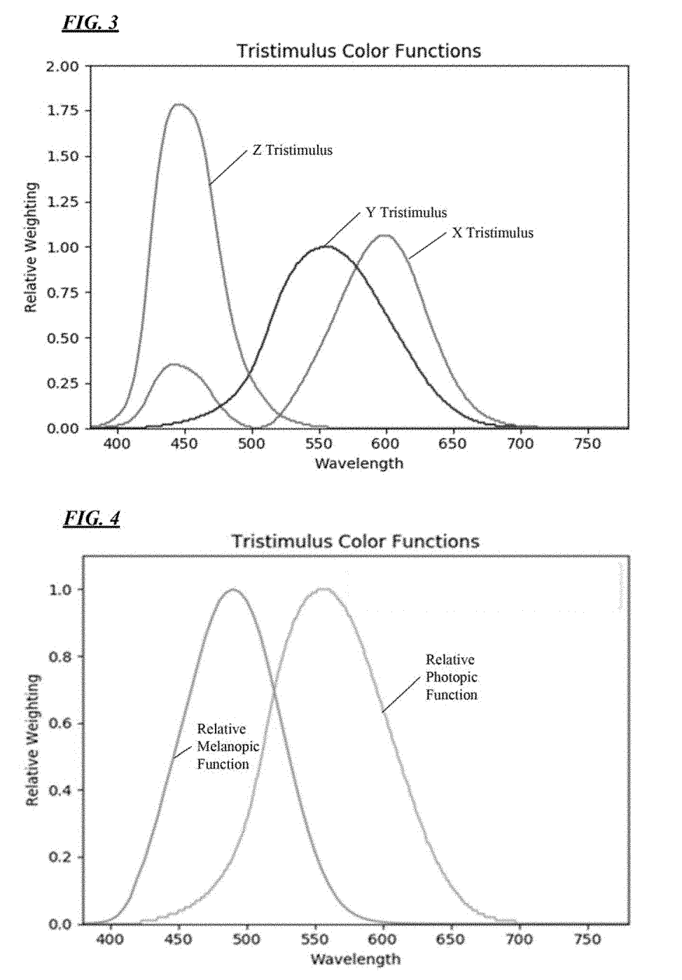

Another method of varying operation based on a variable impact setting and maintaining or optimizing an overall operational parameter is to utilize an aspect of the human visual system called Metamerism where a net spectral power distribution (SPD) from multiple individual spectral outputs of light sources appears the same chromaticity and can be controlled to have the same photometric intensity. For instance, FIG. 2A shows multiple net spectral outputs (Halogen, R/G/B, R/G/B/W, and R/G/B/W/A) which have the same color characteristics of correlated color temperature (CCT) of around 2850 degrees Kelvin, and chromaticity (x,y coordinate) as well as intensity (photopic flux) within tolerance. Each spectrum is comprised of a different quantity and combination of individual light emitters but by controlling each set of emitters differently one is able to maintain these color characteristics via a modification to the overall spectral power distribution. Because of the different sensitivity curves (photopic and melanopic) it is possible to then modify the human stimulus output of the light fixture in a desired way while maintaining intensity and color characteristics. For example, if the light fixture was held to maintain a CCT net output of 2850 Kelvin like in FIG. 2A, utilizing an emitter comprised of Red, Green, Blue, White, and Amber LEDs; one could then switch to only utilizing the Red, Green, and Blue LEDs, compensating with the control levels to output the same intensity and net CCT. The net effect of this on a human biological stimulus like melanopic lux would be an increase, since the blue-ish part of the spectrum now has more radiant power output and closely aligns with the melanopsin sensitivity curve (as discussed more later relative to FIG. 4).

It may be helpful at this point to consider an example technique for calculating photopic and melanopic Lux. FIG. 3 depicts graphs of the tristimulus functions X, Y, and Z. The X, Y, and Z values are mathematical constructs or spectral functions for the color space, which give a translation from the human conal responses to a color space which gives insight into the general perceived hue of a color. The relative photopic sensitivity function was derived before the tristimulus functions, and the tristimulus function Y (generally photopic lumens) was chosen to be the relative photopic sensitivity function. The photopic sensitivity function was derived from human experiments comparing different wavelengths of bright light and finding where they appeared “equally as bright” to a person. FIG. 4 overlays the relative melanopic sensitivity function on the relative photopic function. FIG. 5 is an isolated view of the normalized photopic sensitivity, expressed as a function V(λ) (or V(lambda)) of wavelength of light expressed in in nanometers (nm).

The luminous efficiency function may be expressed mathematically as:

$$φ = 683 ( \frac{lm}{W} ) * {\int }_{0}^{∞} V ( λ ) {φ}_{e} ( λ ) d λ \qquad ( 1 )$$

where:
ϕₑ(λ) is the spectral radiant flux in (W/nm); and
V(λ) is the luminosity function or human photopic sensitivity function.
Note: This calculation is usually done from 380 to 780 nm due to the sensitivity function being so low at those bounds that it is seldom useful to integrate from 0 to infinity.

FIG. 6 graphically depicts a sample spectral power distribution (SPD) curve, such as the SPD curve of an incandescent lamp, with units in the radiant energy per unit area, which may be helpful here since we are talking about melanopic lux as the metric for promoting stimulus impact. The spectrum of the photopic sensitivity curve and melanopsin sensitivity function multiplied by the SPD curve of an incandescent lamp (A) give photopic lux and melanopic lux of 1000 and 547 respectively, as shown in Table 1 below.

TABLE 1

| | | V_Lambda | Melanopsin Sens |
|---|---|---|---|
| Wavelength | SPD (W/m\hat{ }2)-A | (2 degree) | Func |
| 380 | 0.006645947 | 0.000039 | 1.05E−05 |
| 385 | 0.007395347 | 0.000064 | 1.90E−05 |
| 390 | 0.008199841 | 0.00012 | 3.53E−05 |
| 395 | 0.009060854 | 0.000217 | 6.71E−05 |
| 400 | 0.009979335 | 0.000396 | 0.000130339 |
| 405 | 0.010956371 | 0.00064 | 0.000260174 |
| 410 | 0.011992639 | 0.00121 | 0.000526421 |
| 415 | 0.013088684 | 0.00218 | 0.000906471 |
| 420 | 0.014245046 | 0.004 | 0.001565261 |
| 425 | 0.015461795 | 0.0073 | 0.002133928 |
| 430 | 0.016739134 | 0.0116 | 0.00289546 |
| 435 | 0.018076859 | 0.01684 | 0.00365751 |
| 440 | 0.019474698 | 0.023 | 0.004580302 |
| 445 | 0.020932178 | 0.0298 | 0.005406235 |
| 450 | 0.022448687 | 0.038 | 0.0063154 |
| 455 | 0.02402341 | 0.048 | 0.007181519 |
| 460 | 0.0256554 | 0.06 | 0.00807565 |
| 465 | 0.027343568 | 0.0739 | 0.008955784 |
| 470 | 0.029086695 | 0.09098 | 0.009812048 |
| 475 | 0.030883423 | 0.1126 | 0.010467212 |
| 480 | 0.03273226 | 0.13902 | 0.011013205 |
| 485 | 0.034631712 | 0.1693 | 0.011298533 |
| 490 | 0.036579949 | 0.20802 | 0.011405502 |
| 495 | 0.038575205 | 0.2586 | 0.011314512 |
| 500 | 0.040615582 | 0.323 | 0.011017163 |
| 505 | 0.042699178 | 0.4073 | 0.010519279 |
| 510 | 0.044823892 | 0.503 | 0.009841672 |
| 515 | 0.046987619 | 0.6082 | 0.008955981 |
| 520 | 0.049188257 | 0.71 | 0.007979609 |
| 525 | 0.051423497 | 0.7932 | 0.006950765 |
| 530 | 0.053691238 | 0.862 | 0.005922976 |
| 535 | 0.055989104 | 0.91485 | 0.004933259 |
| 540 | 0.058314789 | 0.954 | 0.004011397 |
| 545 | 0.060666052 | 0.9803 | 0.003183677 |
| 550 | 0.063040521 | 0.99495 | 0.002460421 |
| 555 | 0.065435819 | 1 | 0.001848329 |
| 560 | 0.067849708 | 0.995 | 0.001351844 |
| 565 | 0.070280085 | 0.9786 | 0.000962005 |
| 570 | 0.072724031 | 0.952 | 0.000669517 |
| 575 | 0.075179512 | 0.9154 | 0.000456322 |
| 580 | 0.077644492 | 0.87 | 0.00030652 |
| 585 | 0.080116935 | 0.8163 | 0.00020373 |
| 590 | 0.082594128 | 0.757 | 0.000134472 |
| 595 | 0.085074035 | 0.6949 | 8.82E−05 |
| 600 | 0.087555299 | 0.631 | 5.78E−05 |
| 605 | 0.090034527 | 0.5668 | 3.78E−05 |
| 610 | 0.092510363 | 0.503 | 2.48E−05 |
| 615 | 0.09498145 | 0.4412 | 1.63E−05 |
| 620 | 0.097444394 | 0.381 | 1.08E−05 |
| 625 | 0.099898518 | 0.321 | 7.16E−06 |
| 630 | 0.102341786 | 0.265 | 4.77E−06 |
| 635 | 0.104772162 | 0.217 | 3.19E−06 |
| 640 | 0.107188291 | 0.175 | 2.15E−06 |
| 645 | 0.109588135 | 0.1382 | 1.45E−06 |
| 650 | 0.111971016 | 0.107 | 9.87E−07 |
| 655 | 0.114333543 | 0.0816 | 6.75E−07 |
| 660 | 0.116676394 | 0.061 | 4.64E−07 |
| 665 | 0.118996854 | 0.04458 | 3.21E−07 |
| 670 | 0.121294245 | 0.032 | 2.23E−07 |
| 675 | 0.123566532 | 0.0232 | 1.56E−07 |
| 680 | 0.125813035 | 0.017 | 1.09E−07 |
| 685 | 0.128033078 | 0.01192 | 7.70E−08 |
| 690 | 0.130224623 | 0.00821 | 5.46E−08 |
| 695 | 0.132386994 | 0.005723 | 3.89E−08 |
| 700 | 0.13451951 | 0.004102 | 2.78E−08 |
| 705 | 0.136621494 | 0.002929 | 2.00E−08 |
| 710 | 0.13869091 | 0.002091 | 1.44E−08 |
| 715 | 0.140727758 | 0.001484 | 1.05E−08 |
| 720 | 0.142732039 | 0.001047 | 7.63E−09 |
| 725 | 0.144701716 | 0.00074 | 5.58E−09 |
| 730 | 0.146636789 | 0.00052 | 4.10E−09 |
| 735 | 0.148536581 | 0.000361 | 3.03E−09 |
| 740 | 0.150400413 | 0.000249 | 2.24E−09 |
| 745 | 0.152228284 | 0.000172 | 1.67E−09 |
| 750 | 0.154018838 | 0.00012 | 1.25E−09 |
| 755 | 0.155772753 | 0.000085 | 9.35E−10 |
| 760 | 0.15748935 | 0.00006 | 7.03E−10 |
| 765 | 0.159167952 | 0.000042 | 5.31E−10 |
| 770 | 0.160809236 | 0.00003 | 4.03E−10 |
| 775 | 0.162411846 | 0.000021 | 3.07E−10 |
| 780 | 0.163975782 | 0.000015 | 2.34E−10 |
| | 6.4876 | 106.856635 | 1 |
| | | Photopic | |
| | | Lux | Melanopic Lux |
| | | 1000 | 547 |

The equation for the data in Table 1 is similar to equation (1) above where V lambda (2 degree) is multiplied by the SPD in W/m² and integrated over 5 nm increments. The units are in lumens/m² which illustrate illuminance on a surface. It is not common to calculate things this way (in photopic lux); but because the melanopic lux is commonly used as a metric, the data is converted to the photopic sensitivity curve output to the same unit space. Hence, the data for Table 1 is based on the equation:

$${E}_{ v} = 683 ( \frac{lm}{W} ) * {\int }_{0}^{∞} V ( λ ) {E}_{e} ( λ ) d λ \qquad ( 1 A )$$

where Eₑ is spectral irradiance W/m²/nm onto a surface; and
Eᵥ is lumens/m2 (lux).

Note that the SPD in Table 1 is illustrated in (W/m\hat{ }2)—and thus the table calculation does not need to multiply by the differential (5 nm).

The melanopic sensitivity function is a melanopsin photopigment sensitivity function (from the ipRGC photoreceptors) attempting to qualify the sensitivity function and calculate equivalent ipRGC stimulus. This was made in 2014 so has had work done on top of it. This sensitivity curve peaks at 480 (see FIG. 7).

Lucas et al. defined the equation as:

E_{α}=Kₘ∫_{e,λ}(λ)N_{α}(λ)dλ·∫V(λ)dλ/∫N_{α}(λ)dλ (2)

where:
λ is the wavelength of the radiation
Kₘ=683.002 lm/W, the maximum spectral luminous efficacy
V(λ) is the spectral luminous efficacy function for photopic vision
E_{eλ}(λ) is the spectral power distribution
N_{α}(λ) is the α-opic sensitivity curve with normalization.

The normalization was chosen to deliver equivalent melanopic lux as photopic lux for an equal energy emitter. The normalization, however, follows the equation 2 listed above included with the corresponding coefficients.

The N(theta) sensitivity curve for melanopsin is shown in FIG. 7. The preceding equation assumes that V(λ) (or V(lambda)) is the 2 degree observer sensitivity curve shown in FIG. 5, and that Ea(λ) (or Ea(lambda)) in this equation (2) is in W/m\hat{ }2*nm to calculate melanopic equivalent lux. Such a definition differs as it attempts to make melanopic lux and photopic lux equivalent for an equal energy emitter.

The calculation of melanopic lux can be simplified as ∫V(λ)dλ=106.857 and choosing to define ∫V(λ)dλ=1, equation 2 can be simplified to a form equivalent to that for calculations for photopic lux:

E_{α}=72983.25∫E_{eλ}(λ)N_{α}(λ)dλ (3)

Calculating this for the equivalent 1000 photopic lux determined above gives us 547 melanopic lux. It should be noted that melanopic lux is just one current metric for the impact of light to our circadian system and biology and there are other metrics that may be used for stimulus impacts.

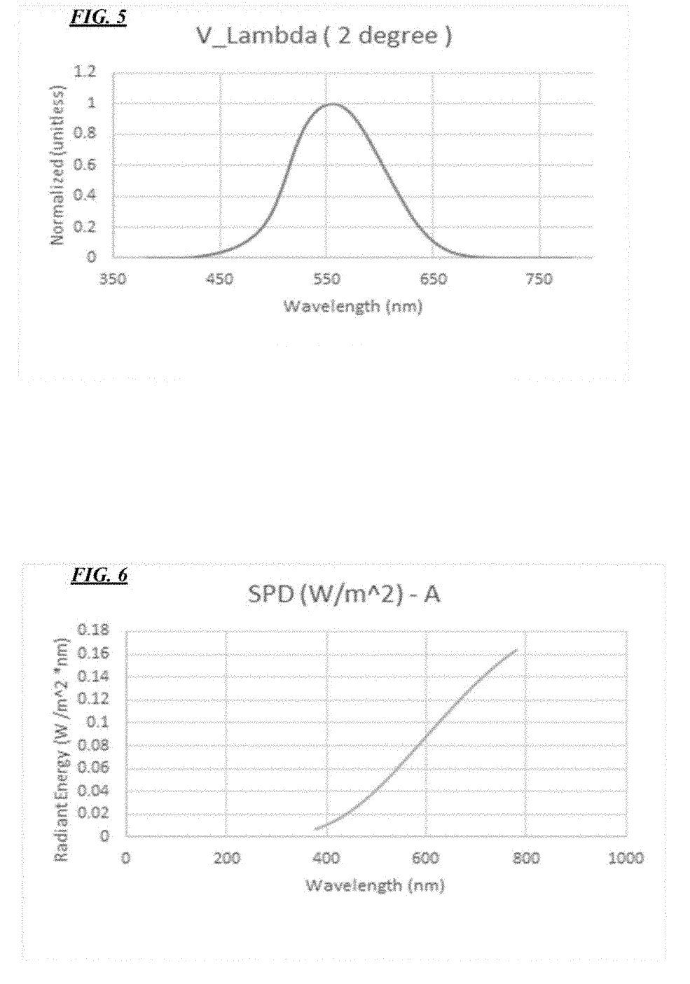

Assuming that the array in the light source 110 has red/green/blue LEDs at the pixels of the array, the spectral power distributions of the individual LEDs in the emitters of the array may be as shown in FIG. 8.

We can then calculate the total net illuminance by applying the photometric curve to all three LEDs as follows:

$$Ev = 683 ( \frac{lm}{W} ) * [ {\int }_{0}^{∞} V ( λ ) φ {R}_{e} ( λ ) d λ + {\int }_{0}^{∞} V ( λ ) φ {G}_{e} ( λ ) d λ + {\int }_{0}^{∞} V ( λ ) φ {B}_{e} ( λ ) d λ ] \qquad ( 4 )$$

where ϕ(λ), the PHI-COLOR(lambda), is in W/m²*nm (spectral irradiance)

Then, similarly for melanopic lux, we can calculate:

Eα=72983.25*[∫0∞N_{α}(λ)ERₑ(λ)dλ+∫₀^{∞}N_{α}(λ)EGₑ(λ)dλ+∫₀^{∞}N_{α}(λ)EBₑ(λ)dλ] (5)

From these computations, an increase melanopic lux could be achieved while simultaneously maintaining lumen output. One way to do this would to be to increase the blue channel while simultaneously reducing the red and green channels in a non-uniform way to maintain lumen output while increasing blue for more melanopic lux. This simple approach would change the net power input and change the color rendition and CCT of the overall light output from the light source 110.

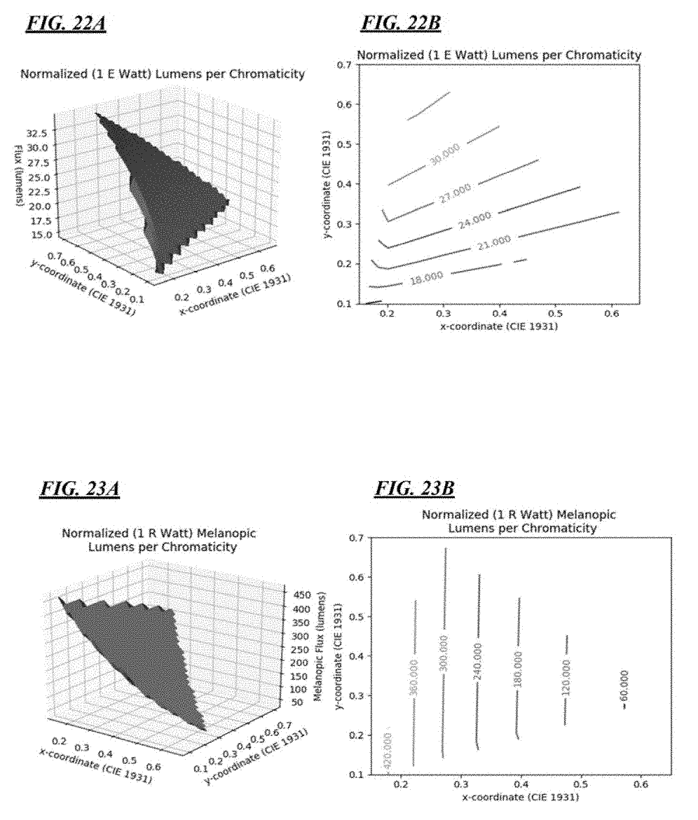

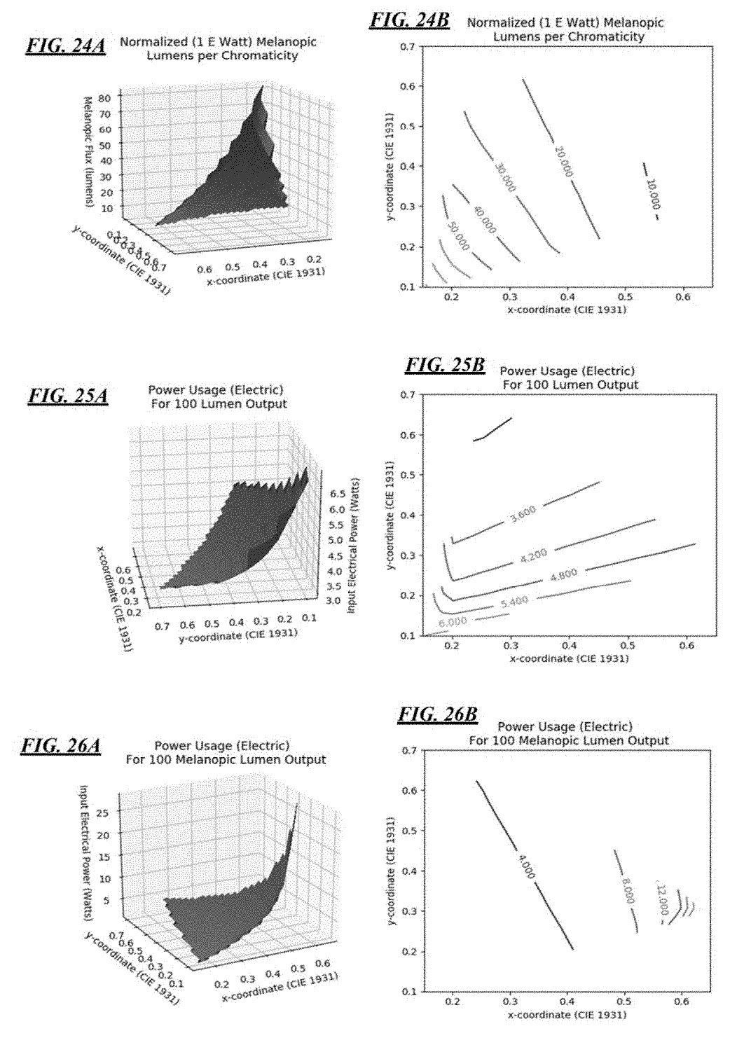

Any given chip emission spectrum will have a ratio of overall radiometric output power to both photopic lux and melanopic lux. The spectrums scale linearly relative to any increase or decrease in radiometric power. Hence, in the preceding example an increase in blue and a decrease in red is an example of a non-uniform modification to effectively increase the melanopic/photopic ratio. The surface plot of this ratio for the example is shown in FIGS. 21A and 23A. FIG. 8A shows an example of the different relationships of outputs for photopic lux and melanopic lux for red, green and blue emitter chips.

The optimization may be implemented in a variety of ways. For example, one approach involves selecting an image for display that achieves a desired stimulus impact, then processing the image to maintain a desired general illumination level (e.g. overall intensity level) within some tolerance yet maintain the desire stimulus impact. The image quality may change, by the stimulus impact and the general illumination performance can still be sufficiently achieved. This and several other optimization examples are described in greater detail later.

At a high level, the optimization examples involve categorizing the spectrum of individual LEDs or similar individual emitters as the output channels of the source array in several dimensions. The examples, at a minimum use two LEDs or the like as a combined emitter at each pixel point of the array. Additionally the intention of the examples generally is to meet a specific melanopic lux or other stimulus impact value and then adjust for light output, power requirements or other operational parameter as an optimization parameter. There is an initial bounding metric of numerical circadian stimulus or melanopic lux due to overall light emitter limitations, and then an optimization parameter such as net CCT, net power usage, or net light output. Depending on the amount of LEDs utilized there can be a cascading quantity of optimized and dependent variables. The term optimization here could be multi-variable such as “maintain CCT as close as possible without going over X watts”. Some systems would be better at optimizing than others such as using a two different characteristic white channels plus a blue channel solution.

The examples encompass a lighting device which has individual pixel control of many emitters in the array of light source 110. Each emitter for a pixel may have a multitude of individual channels (individual channels within the pixel emitter), such as RGBW or warm white/cool white, and many other types.

Using a lighting device with such pixel-level control, a representational image (blurred and abstract) may be adjusted to maintain the macro look of the image but modified in spectrum to keep a relative difference between the pixels defined by an algorithm to simultaneously hit the variables described herein. In addition to this image processing, an effect could be done by a pseudo-random algorithm which takes in a style of generating organic feeling shapes in a random way such as blobs of ink spreading through water, and then non-randomly simultaneously optimizes the operational parameters algorithmically by changing the outputs of the channels of the LED pixels. A control input parameter for this lighting device could be a numeric of the user's choosing. Another variation of the pseudo-algorithm could be to interlace or tessellate shapes which also gets fed into a circadian stimulus solving algorithm while optimizing one or more of the above metrics. All of the above would utilize information on the LEDs or other types of individual channel emitters used in the pixel emitters of the array, such as spectrum, power, and lumen output curves.

At this point, it may be helpful to consider a more detailed example of a lighting device and a luminaire having such a light source.

FIG. 9 illustrates an example of a lighting device 100, in high level block diagram form. As a general overview, lighting device 100 in the example includes a luminaire 105 having a light source 110, and includes a controller 150. The image or algorithm based light output of the luminaire 105 may change the biological stimulus impact based on a control signal, time of day, a sensor signal, feedback from a cloud controller or the like. General illumination parameters, such as ON/OFF state, desired output intensity and the like may similarly be based on a control signal, time of day, a sensor signal, feedback from a cloud controller or the like. Additional details regarding lighting device 100 are set forth below.

The luminaire 105 includes a light source 110, typically in the form of an emitter matrix or array of independently controllable light emitters. Each respective light emitter is configured to be controlled to vary intensity or a color characteristic of light emitted from the respective light emitter. The emitters may be considered as emitters at pixel points of the array in that each such emitter is located and controlled to emit light of a pixel of the current image intended for output from the luminaire 105, from a point of the array/matrix. The individually controllable light emitters of the array together are configured to be able to operate within tolerance of a predetermined level of a light source operations parameter. The parameter may be a parameter of combined emitted light to be output from the lighting device, such as a particular intensity and/or a particular spectral characteristic. Operation of the light source may be controlled to cause the emitters of the source to maintain operation within tolerance of other types of operational parameters, e.g. within some percentage of a target for overall power consumption.

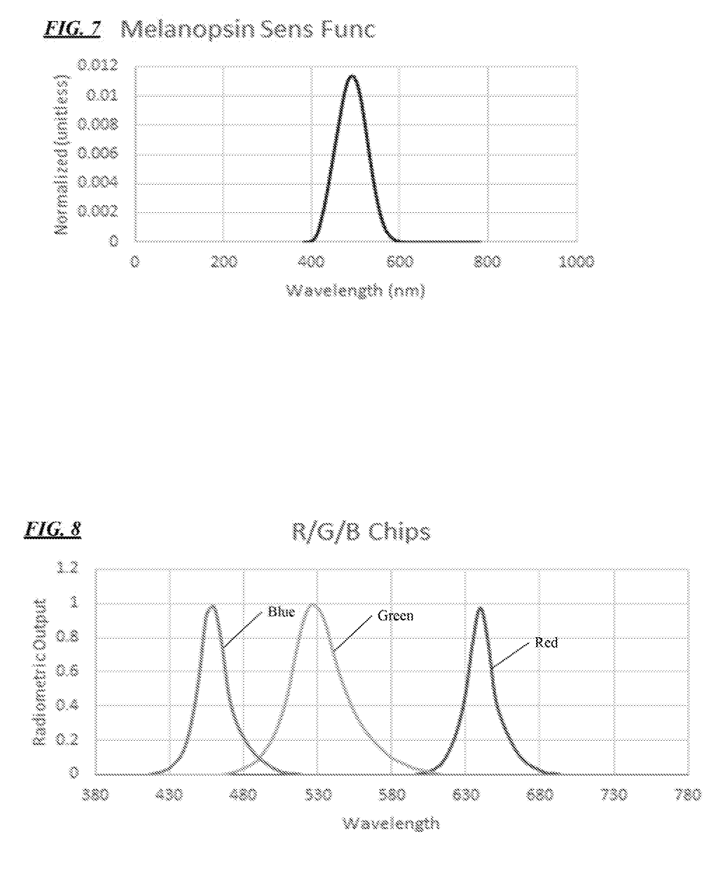

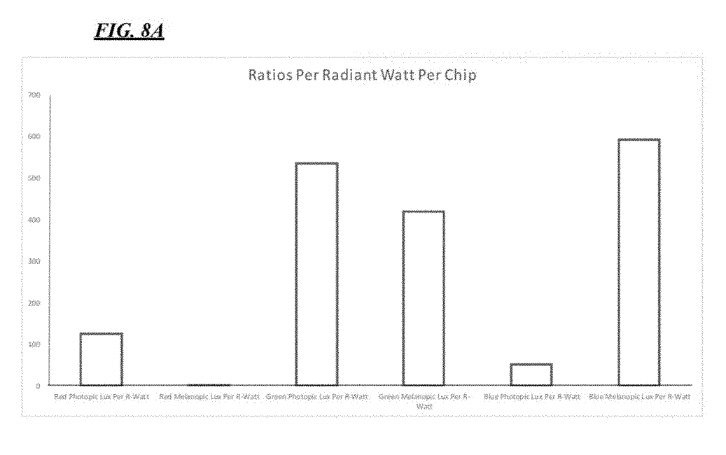

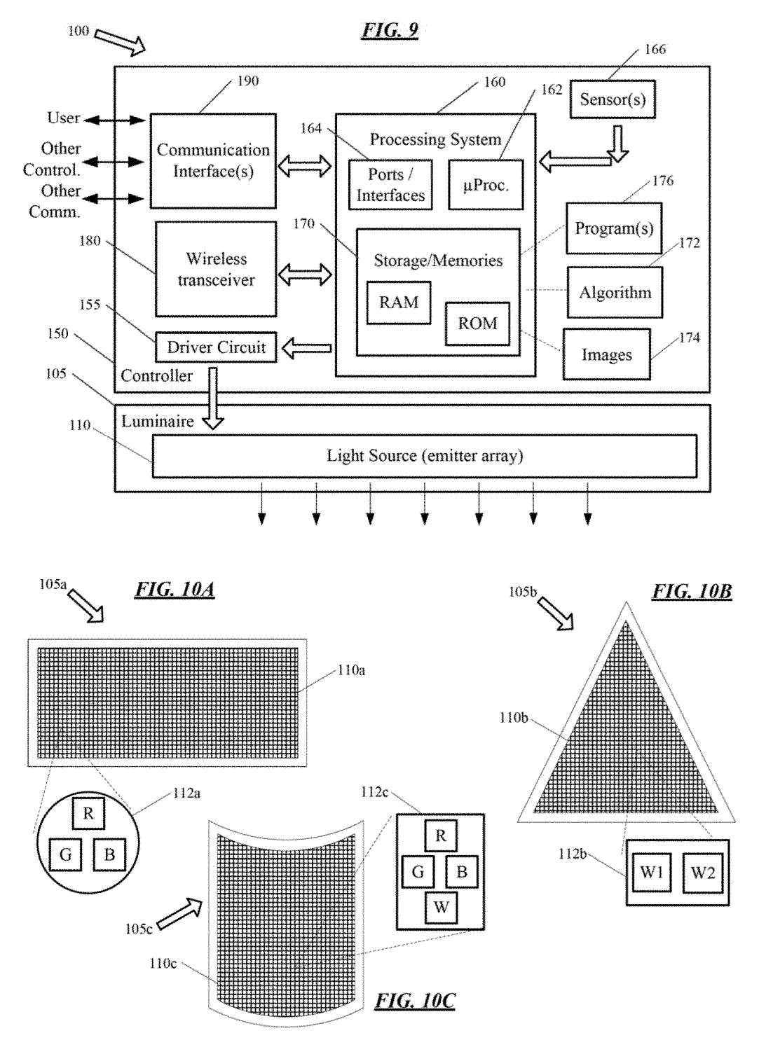

FIGS. 10A to 10C are plan views of several example configurations 105a to 105c of luminaires like 105 (FIG. 9) and arrays used as light sources 110a to 110c in such luminaires. FIGS. 10A to 10C also provide enlarged views of several different examples of individually controllable pixel point emitter configurations.

The luminaire 105 may be implemented in a wide range of different form factors. In FIG. 10A, the luminaire 105a is rectangular and has a rectangular light emitter array 110a. FIG. 10B depicts a triangular shaped luminaire 105b and correspondingly configured light emitter array 110b. The luminaire 105b and emitter 110b may be relatively flat or somewhat curved or contoured (in the dimension perpendicular to the plane of the drawing). FIG. 10C represents a luminaire 105c with straight sides but curved ends, and the light emitter array 110c is similarly configured. Again, the luminaire 105c and array 105c may be relatively flat or somewhat curved or contoured (in the dimension perpendicular to the plane of the drawing). These views are intended as just a few representative examples.

The illumination control techniques under consideration here take advantage of capabilities of multi-channel control and light emission of suitable emitters. In the examples, each independently controllable emitter at a pixel point of an array has at least two controllable channels and individual emitters associated with those channels capable of emitting light of corresponding different spectral characteristics. The individual emitters in a pixel point emitter group may emit light of different primary colors, white light of different color temperatures, white light plus light of one or more primary colors, etc.

FIG. 10A, for example, shows a group of individual emitters 112a at a pixel point of the array 110a forming the light source, in which the group includes red (R), green (G), blue, (B) light emitting diode (LED) type emitters. Controlled mixing of the RGB lights at different relative intensities from those LEDs allows a wide range of color tuning, including a range of different types of white light (with different spectral characteristics).

FIG. 10B shows a group of individual emitters 112b at a pixel point of the array 110b forming the light source, in which the group includes two different white (W) type LEDs emitting white light of two different spectral characteristics (e.g. identified by different CCT values). LED W 1, for example, may emit white light of 4500° K, whereas LED W2 may emit white light of 2700° K. Controlled mixing of the W1, W2 lights at different relative intensities allows tunable white light output in a range roughly from 2700° K to 4500° K.

FIG. 10C shows a group of individual emitters 112b at a pixel point of the array 110b forming the light source, in which the group includes RGBW LEDs. Controlled mixing of the RGBW lights at different relative intensities allows a wide range of color tuning, including a range of different types of white light. The inclusion of the white (W) LED adds extra intensity for white light output, or could act as an additional channel to maintain specific color characteristics and intensity while changing desired human stimulus.

The LEDs in the pixel points may be integrated on a single chip or provided as two or more individually packaged emitter devices. LEDs are used by way of examples, but other controllable light emitters, for example, other organic or inorganic semiconductor light emitters, plasma devices, LCD based sources, may be used. Also, the three pixel point emitter configurations are given by way of examples, only. Other arrangements with different color emitters and/or with different numbers of individual emitters per pixel point may also be used in a luminaire 105. Although the emitter examples are shown in association with particular luminaire/array configurations in FIGS. 10A to 10C, that association is by way of example only; and these and other emitter configurations may be used in each of the example luminaire/array configurations or in other luminaire/array configurations.

A luminaire 105 like any of those shown in FIGS. 9 to 10C is not size restricted. For example, each luminaire 105a may be of a standard size, e.g., 2-feet by 2-feet (2×2), 2-feet by 4-feet (2×4), or the like, and arranged like tiles for larger area coverage. Alternatively, a luminaire 105 may be a larger area device that covers a wall, a part of a wall, part of a ceiling, an entire ceiling, or some combination of portions or all of a ceiling and wall.

Returning more specifically to the device example of FIG. 9, the drawing illustrates an example of a controller 150 that may be used in lighting device 100. Controller 150 is coupled to control light source 110 to provide general illumination and the associated controlled human stimulus impact. The processing system 160 provides the high level logic or “brain” of the lighting device 100. In the example, the processing system 160 is optionally coupled with one or more sensors 166, a wireless transceiver 180 and communication interface(s) 190.

In one implementation example, the controller 150 of the lighting device 100 includes a driver circuit 155 that is coupled to the light source 110 in the luminaire 105 to control light outputs generated by the emitter array in the light source 110. Although the driver circuit 155 is implemented as an element of the controller 150, the driver circuit 155 may be separately located from other elements of the controller 150, for example, in the luminaire 105. In the examples, the light source 110 is formed by a multi-pixel array or matrix of independently controllable light emitters, such as color characteristic and intensity controllable LED based pixel emitters. For such device implementations, the driver circuit 155 may be a matrix type driver circuit, such as an active matrix driver or a passive matrix driver. Multiple-emitter capable pulse-width modulation driving circuitry also may be adequate to tune the light intensity and/or spectral characteristic of LED emitters at the pixels of some implementations of the array of the source 110, for example, implementation intended for outputs of low resolution and/or relatively slow dynamic variation over time.

Processing system 160 includes a central processing unit (CPU), shown by way of example as a microprocessor (μP) 162, although other processor hardware circuitry may serve as the CPU. Processing system 160 also includes memory or other types of storage 170, which may include a random access memory and/or a read-only memory or the like. The CPU and storage/memories, for example, may be implemented by a suitable system-on-a-chip often referred to as a micro-control unit (MCU). In a microprocessor implementation, the microprocessor may be based on any known or available microprocessor architecture, such as a Reduced Instruction Set Computing (RISC) using ARM architecture, as commonly used today in mobile devices and other portable electronic devices. Of course, other microprocessor circuitry may be used to form the processor 162 of the controller 150. The processor 162 may include one or more cores. Although the illustrated example includes only one microprocessor 162, for convenience, a controller 150 may use a multi-processor architecture, for example, in an implementation in which the host processing system 160 is configured to perform complex image processing or algorithm processing or in an implementation in which one host processing system 160 is intended to control a large number of luminaires 105. Although not shown, the controller 150 may include additional or alternative types of processors, such as an arithmetic logic unit, a dedicated image processor, an image data decoder, etc.

Processing system 160 also includes a ports and/or interfaces 164. The ports and/or interfaces 164 couple the microprocessor 162 to various other elements of the lighting device 100, such as the driver circuit 155, one or more sensors 166 (such as motion or thermal sensors), the wireless transceiver 180, and/or the communication interface(s) 190. In a microprocessor based implementation, the ports and/or interfaces 164 may be suitable interface devices connected to an internal bus or the like of the system 160, which also connects to the processor 162 and the memory 170. In an MCU type implementation of the processing system 160, ports and/or interfaces 164 would be integrated on the system-on-a-chip with and internally connected to the processor 162 and the memory 170.

The processor 162, for example, by accessing programming 176 in the memory 170, controls operation of the driver circuit 155 and thus operations of the luminaire 105 via one or more of the ports and/or interfaces 164. In a similar fashion, one or more of the ports 164 enable processor 162 of the processing system 160 to use and communicate externally via communication interface(s) 190; and one or more of the ports 164 enable processor 162 of the processing system 160 to receive data regarding any condition detected by a sensor 166, for further processing.

As noted, the host processor system 160 is coupled to the communication interface(s) 190. In the example, the communication interface(s) 190 offer a user interface function or communication with hardware elements providing a user interface for the lighting device 100.

The communication interface(s) 190 also or instead may communicate with other control elements, for example, a host computer of a building control and automation system (BCAS). The communication interface(s) 190 may also support device communication with a variety of other equipment of other parties having access to the lighting device 100 in an overall/networked lighting system encompassing a number of lighting devices 100, e.g. for access to each lighting device 100 by equipment of a manufacturer for maintenance or access to an on-line server for downloading of programming instruction or configuration data for setting aspects of luminaire operation.

External communication by communication interface(s) 190, or communication within the internal components of lighting device 100, may be accomplished by any known manner of communication, including electrical communication, optical communication (such as visible light communication (VLC) or fiber optic communication), electromagnetic communications, or others.

As another example, processing system 160 may operate a wireless transceiver 180 (if included) to communicate information to or from a wireless device in the area illuminated by light source 110. Wireless transceiver 180 may be a personal area network (PAN) transceiver, a transceiver operating in accordance with Bluetooth or Bluetooth Low Energy communication standards, a WiFi transceiver, an ultra-wide band (UWB) transceiver, or the like. Such communications, for example, may allow a user to configure or control operations of the lighting device 100.

Controls related to coordination of variation for stimulus impact while achieving or maintaining a desirable light source operational parameter may be implemented by program instructions, setting data or a combination of program instructions and data, which are stored in the storage/memories 170. Some implementations utilize an algorithmic approach, which would be controlled by program instructions and/or data related to the algorithm, shown for convenience at 172 in FIG. 9. Other approaches select among a number of stored images shown at 174. Other approaches involve processing a stored image from 174, based on algorithm instructions 172 or based on other programming (not separately shown).

Apparatuses implementing functions like those of configurable lighting device 100 may take various forms. For example, a lighting device 100 may have all of the above hardware components on or within a single hardware platform as generally shown in FIG. 9, or some components attributed to the lighting device 100 may be separated from the luminaire 105 with the light source 110, in different somewhat separate units. In a particular example using separate units, one set of the hardware components of some or all of the controller 150 may be separated from one or more instances of the controllable luminaire 105, e.g. such that one host processor system 160 may control several luminaires 105 each at a somewhat separate location. In such an example, one or more of the controlled luminaires 105 are at a location remote from the one host processor system 150. In such an example, a driver circuit 155 may be located near or included in each luminaire 105. For example, one set of intelligent components, such as the microprocessor 123, may control/drive some number of driver circuits 155 and associated controllable luminaires 105. Alternatively, there may be one overall system of one or more driver circuits 155 located at or near the host processor system 160 for driving some number of luminaires 105. It also is envisioned that some lighting devices may not include or be coupled to all of the illustrated elements, such as the sensor(s) 166, the transceiver 180 and/or the communication interface(s) 190. For convenience, further discussion of the lighting device 100 of FIG. 9 will assume an intelligent implementation of the lighting device 100 that includes at least the illustrated components.

As noted, a lighting device providing illumination and associated human stimulus impact may be implemented as a fairly standalone device (as in FIG. 9) or in a networked system of such devices. It may be helpful to consider a high-level example of a system including a number of implementations of lighting devices 100, with reference to FIG. 11. For that purpose, FIG. 11 illustrates a lighting system 200 for providing lighting for general illumination or the like in a space 213 at a premises 215 and for varying lighting in a manner to promote one or more intended human stimulus impacts. The system 200 may also enable communication of configuration or setting information, including with respect to intended human stimulus impacts, to at least one lighting device (LD) 100 of any of the types discussed herein.

The system example 200 shown in the drawing includes a number of such lighting devices (LD) 100. For purposes of discussion of FIG. 11, it is assumed that each lighting device 100 generally corresponds in structure to the block diagram illustration of a lighting device 100 in FIG. 9, with the illumination light source 110 and structured/located to operate as a luminaire 105 as discussed in various other examples herein. The example of the lighting system 200 in FIG. 11 also includes a number of other devices or equipment configured and coupled for communication with at least one of the lighting devices 100.

In the lighting system 200 of FIG. 11, the lighting devices 100, as well as some other elements of system 200, are installed within the space or area 213 to be illuminated at the premises 215. The premises 215 may be any location or locations serviced for lighting and other purposes by such a system 200 of the type described herein. Lighting devices, such as lighting devices 100, that are installed to provide general illumination lighting in the premises 215 typically comply with governmental building codes (of the respective location of the premises 215) and/or lighting industry standards. Most of the examples discussed herein focus on indoor building installations, for convenience, although the system may be readily adapted to outdoor lighting. Hence, the example of lighting system 200 provides controllable lighting (e.g. for general illumination and for stimulus impact) and possibly other services in a number of service areas in or associated with a building, such as various rooms, hallways, corridors or storage areas of a building and an outdoor area associated with a building. Any building forming or at the premises 215, for example, may be an individual or multi-resident dwelling or may provide space for one or more enterprises and/or any combination of residential and enterprise facilities. A premises 215 may include any number of such buildings, and in a multi-building scenario the premises may include outdoor spaces and lighting in areas between and around the buildings, e.g. in a campus (academic or business) configuration.

The system elements, in a system like lighting system 200 of FIG. 11, may include any number of lighting devices 100 as well as one or more lighting controllers 219. The lighting controller 219 may be an automated device for controlling lighting, e.g. based on timing conditions; and/or the lighting controller 219 may provide a user interface. Lighting device controller 219 may be configured to provide control of lighting related operations (e.g., ON/OFF, intensity or brightness, color characteristic(s), etc.) of any one or more of the lighting devices 100. A lighting controller 219, for example, may take the form of a switch, a dimmer, or a smart control panel including a graphical, speech-based and/or touch-based user interface, depending on the functions to be controlled through the device 219.

A lighting device 100 may include a sensor (as in FIG. 9). In the example, other system elements may also include one or more standalone implementations of sensors 212. Sensors, for example, may be used to control lighting functions in response to various detected conditions, such as occupancy or ambient light. Other examples of sensors include light or temperature feedback sensors that detect conditions of or produced by one or more of the lighting devices. If separately provided, the sensors may be implemented in intelligent standalone system elements such as shown at 212 in the drawing. Alternatively, sensors may be incorporated in one of the other system elements, such as one or more of the lighting devices 100 and/or the lighting controller 219.

The on-premises system elements 100, 212, 219, in a system like the system 200 of FIG. 11, are coupled to and communicate via a data network 217 at the premises 215. The data network 217 may be a wireless network, a cable network, a fiber network, a free-space optical network, etc.; although the example shows connection lines as may be used in a hard-wired or fiber type network implementation. The data network 217 in the example also includes a wireless access point (WAP) 221 to support communications of wireless equipment at the premises (e.g. for an installation in which none of the lighting devices 100 includes a wireless transceiver 190 or an installation in which the network 217 provides more general data communication services at the premises 215. For example, the WAP 221 and network 217 may enable a user terminal for a user to control operations of any lighting device 100 at the premises 213 and/or to access an external data network 223, such as the Internet. Such a user terminal is depicted in FIG. 11, for example, as a mobile device 225 within premises 215, although any appropriate user terminal may be utilized.

However, the ability to control operations of a lighting device 100 or group of such devices 100 may not be limited to a user terminal accessing data network 217 via WAP 221 or other on-premises point of access to the network 217. Alternatively, or in addition, a user terminal such as laptop 227 located outside premises 215, for example, may provide the ability to control operations of one or more lighting devices 100 via one or more other networks 223 and the on-premises data network 217.

Network(s) 223 may include, for example, a local area network (LAN), a metropolitan area network (MAN), a wide area network (WAN) or some other private or public network, such as the Internet.

Data network communications allow installation of programming, data of one or more images, setting data for operational parameters such as illumination attributes or for stimulus impacts, etc. Such data communications also may allow selection among installed image data files or installed setting files in any lighting device 100 that stores more than one such image or setting file. In another example, a memory device, such as a secure digital (SD) card or flash drive, containing image data, setting data or the like may be connected to one or more of the on-premises system elements 100, 212 or 219 in a system like system 200 of FIG. 11 for upload thereof into a lighting device 100 or other system elements 212 or 219.

For lighting operations, the system elements (100, 212 and/or 219) for a given service area 213 are coupled together for network communication with each other through data communication media to form a portion of a physical data communication network 217. Similar elements in other service areas of the premises 215 are coupled together for network communication with each other through data communication media to form one or more other portions of the physical data communication network 217 at the premises 215. The various portions of the network 217 in the service areas in turn are coupled together to form a data communication network at the premises, for example to form a LAN or the like, as generally represented by network 217 in FIG. 11. Such data communication media may be wired and/or wireless, e.g. cable or fiber Ethernet, Wi-Fi, Bluetooth, or cellular short range mesh. In many installations, there may be one overall data communication network 217 at the premises. However, for larger premises and/or premises that may actually encompass somewhat separate physical locations, the premises-wide network 217 may actually be built of somewhat separate but interconnected physical networks utilizing similar or different data communication media.

System 200 also includes a server 229 and a database 231 accessible to a processor of server 229. Although FIG. 11 depicts server 229 as located outside premises 215 and accessible via network(s) 223, this is only for simplicity and no such requirement exists. Alternatively, server 229 may be located within premises 215 and accessible via network 217. In still another alternative example, server 229 may be located within any one or more system element(s), such as lighting device 100, lighting controller 219 or sensor 212. Similarly, although FIG. 11 depicts database 231 as physically proximate server 229, this is only for simplicity and no such requirement exists. Instead, database 231 may be located physically disparate or otherwise separated from the server 29 and logically accessible by a processor of the server 229, for example via network 217 and/or 223.

Database 231 in this example is a collection of files for use in conjunction with one or more of lighting devices 100 in premises 215 and/or similar devices 100 of the same or other users in other areas or at other premises. The files, for example, may contain data of images, illumination settings, impact settings, input values for stimulus impact algorithms, algorithm formulas, program instructions, etc. Each lighting device 100 in the system is configured to receive some of the information from the database 231 to configure the lighting device and perform operations as described herein.

As outlined earlier, there are a number of techniques to operate a lighting device 100 alone or as part of a lighting system 200, so that each lighting device outputs light so as to promote a desired or intended stimulus impact. It may be helpful to consider several examples of such techniques in more details. Although the following examples may be applied to other stimulus factors and/or utilize other stimulus metrics, for convenience, the illustrations and discussions of the following examples will concentrate on examples relating to a circadian stimulus impact as specified by a setting or metric value expressed as melanopic lux.

Several of the procedural examples involve output of light representing a specific image from a light source 110 of a luminaire 105. There are various was to process image data to adapt the image light output from the source 110 to promote stimulus impact and achieve or maintain operation of the light source 110 within tolerance of a setting value for an operational parameter of the light source.

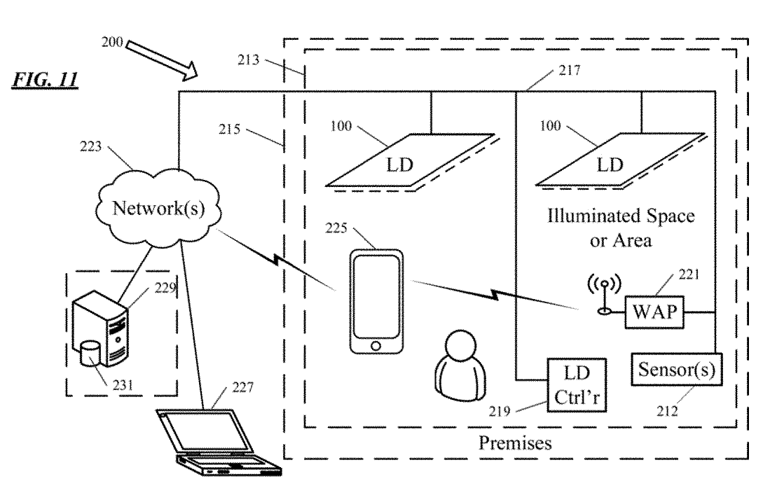

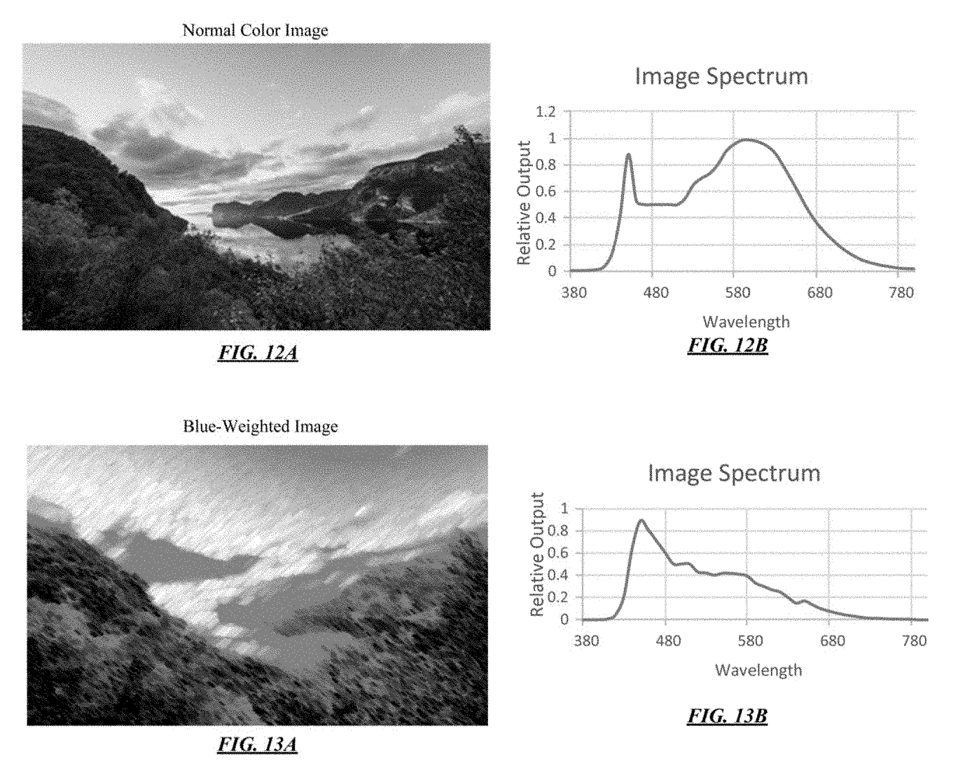

A first image based approach is illustrated generally in FIGS. 12A to 13B, involving image data processing to shift spectral power distribution to achieve a desired melanopic lux setting.

FIG. 12A shows a sample image. Although shown as a grey-scale image for convenience, in practice the image typically would be a full color image. FIG. 12B shows a spectrum distribution graph for the sample color image. The data of the image of FIG. 12A may be used to produce drive signals for the individual LEDs of the pixel level emitters of the array of light source 110, which in turn will output light with a spectral power distribution approximating that shown in FIG. 13B. For discussion purpose, assume that the light output corresponding to FIG. 13B is an initial image output from the source 110 and has a relatively low level of melanopic lux, e.g. to deliver a melanopic lux of 100.

At a particular time of day, a processor of or in communication with the lighting device 100 obtains a new desired stimulus impact setting, in this case, indicating an increase to a new higher value of melanopic lux. For discussion purposes, assume that the new setting value is a value of 200 for melanopic lux. As outlined earlier, the blue included in light output may be increased to achieve a higher value of melanopic lux.

A processor of or in communication with the lighting device 100 may process data of an image, such as the image of FIG. 12A, to shift the output spectrum distribution to provide a higher amount of blue light. In the example, the grey-scale version of the image in FIG. 13A shows changes form the image in FIG. 12A intended to indicate that the image in FIG. 13A is bluer than the image in FIG. 12A. The data of the original image (see FIGS. 12A and 12B) has been filtered or otherwise processed to increase the blue content of the output light of the image.

Since the melanopic sensitivity function weights blue higher, the blue-weighted distribution of FIG. 13B provides a specified higher amount of melanopic lux, e.g. to deliver a melanopic lux of 200. In this way, the data of the image can be processed and shifted towards the sensitivity of the desired function to elicit a more desired response to the melanopic stimulus factor.

The image in the example of FIGS. 12A and 12B represented a relatively clear image presentation, e.g. as might be perceived as a still of moving picture when output from a rectangular emitter array. The resolution of the image in the light output of the light source 110 would depend on the resolution of the emitter array of the source. The present concepts also apply to outputs of more representational images that may not readily appear like may be created by selecting all of a part of a picture type image and blurring the data of the selection to the point that the light output is not readily recognizable as a part of a picture.

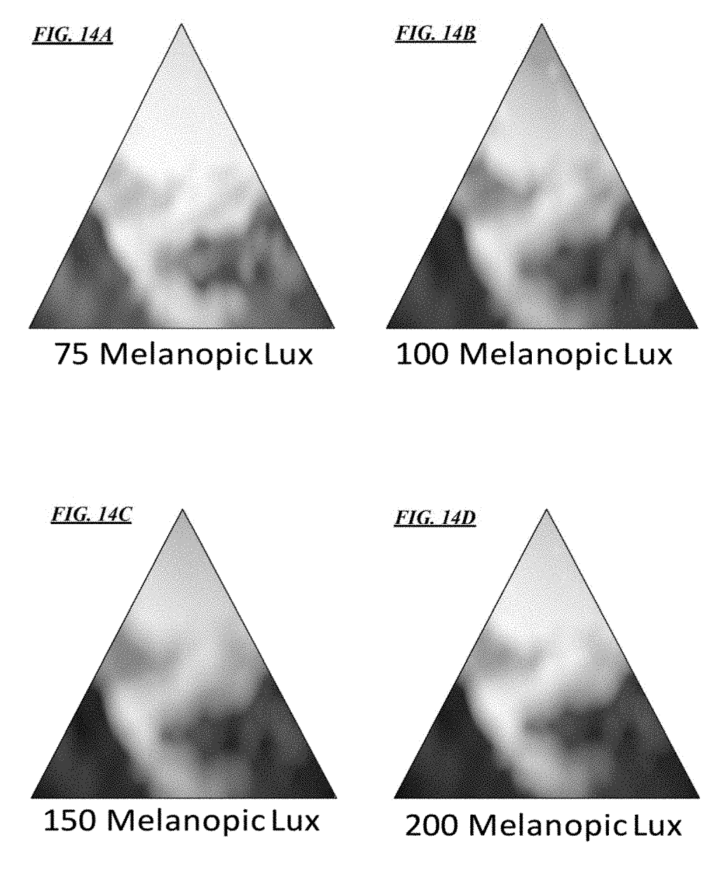

FIGS. 14A to 14D are plan views of luminaire output light based on a portion of an image, where the image data has been processed to provide four different values of melanopic lux. In these examples, the luminaire 105 and its light source array 110 are triangular, like in FIG. 10B. Also, the base image is a representational image, for example, formed by blurring a selected portion of an image like that of FIG. 12A, which is expanded and effectively overlaid on the triangular emitter array output via image data processing and conversion to appropriate emitter drive signals.

The illustration in FIG. 14A is intended to represent a somewhat yellowish image light output, at a setting of 75 melanopic lux. The illustration in FIG. 14B is intended to represent a somewhat greenish image light output, at a setting of 100 melanopic lux. The illustration in FIG. 14C is intended to represent a slightly blueish image light output, at a setting of 150 melanopic lux; and the illustration in FIG. 14D is intended to represent a predominantly blueish image light output, at a setting of 200 melanopic lux.

In operation, a processor might respond to a time of day schedule to control the luminaire 105 to output light of the different images of FIGS. 14A to 14D to provide different melanopic lux settings corresponding to the different times of day. The data representing the different images may be pre-stored or may be based on real-time processing of data for a base image at the appropriate times of day.

The image data processing in examples like those of FIGS. 12A to 14D may utilize a variety of aspects of image modification which. For example, highlights of an image could be raised/lowered to change resultant melanopic lux. As another example, a processor could apply a virtual filter overlaid on the data of the image to enhance blue or amber (like a virtual gel). In another approach, the image data may be processed to increase or decrease the brightness level of the image light output from the luminaire. By way of yet another example, portions of the data of the image may be modified to correspond to black/white outputs at some selected pixels. In another example, the whole output of the light source 110 or a specific subset of the output could be modified, for instance, the bluish aspect and thus contribution to melanopic lux provided the sky shown in an image could be selectively reduced to bring the aggregate of the entire spectral power distribution (SPD) to a specific output level.

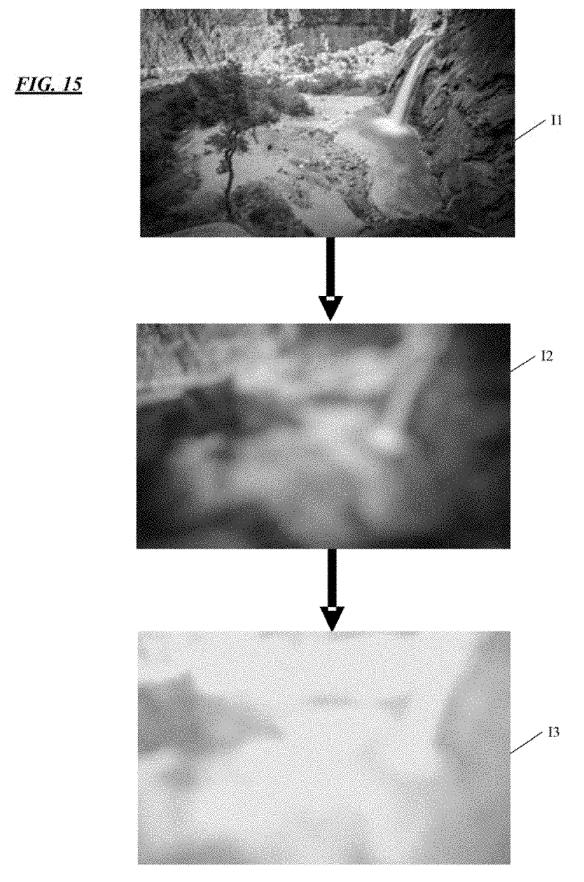

FIG. 15 depicts an image processing flow in the form of three progressive states of an image, representing light output states to promote different degrees of human stimulus impact, as might represent an example of one such image processing technique. As shown, an image at I1 is analyzed and modified in such a way where highlights are enhanced at I2 and white balance is modified to get to the image I3, which provides a specific lumen target output while also maintaining the biological effect.

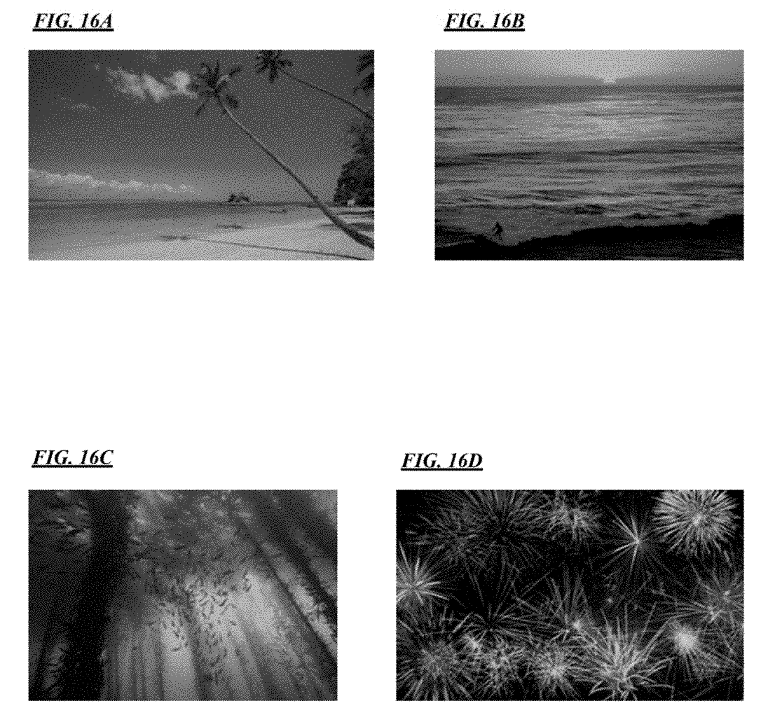

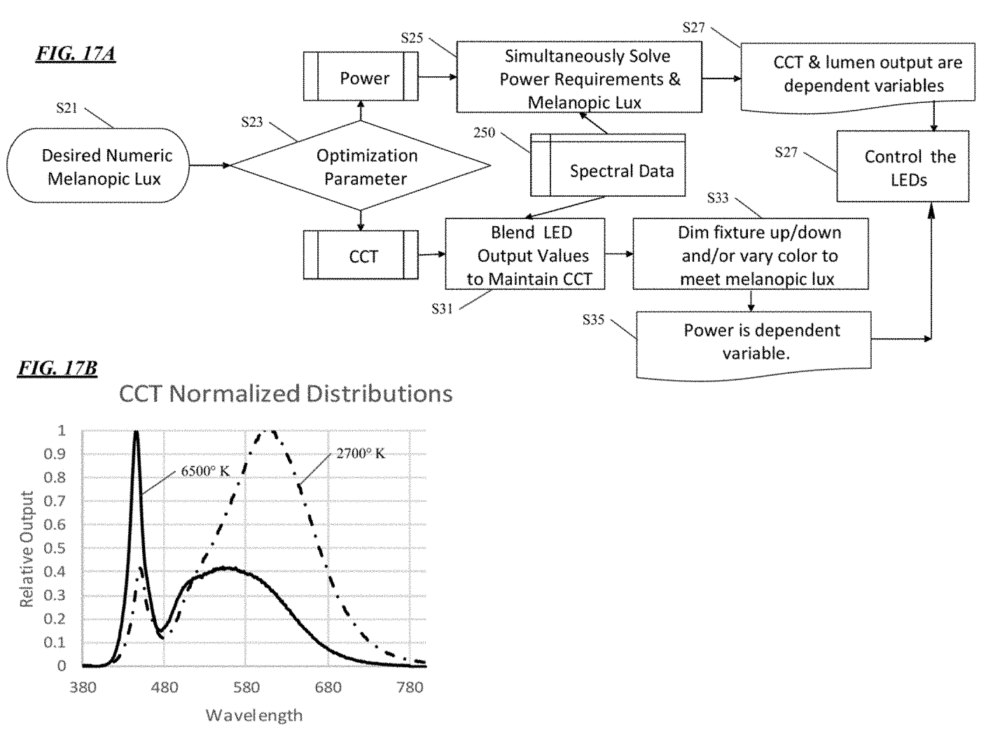

FIGS. 16A to 16D depict by way of example several images of different characteristics that may be selected for output to promote different degrees of human stimulus impact. The approach represented by these example images involves library image selection. The lighting device 100 could have an internal or cloud accessible library of data files for images that have a circadian impact associated therewith. Image files may also be associated with themes.

With this approach, a processor may choose one or several files for images to use as a static or series of images or even a video. The set of images could be selected based on a stimulus impact metric (i.e. circadian impact or melanopic lux values) or by a user theme (beach scenes) or both. The lighting device (based on direct user input, time of day, sensed condition, etc.) could select the biological input setting desired (e.g. a setting corresponding to a high circadian impact), and the user might also select an image category (e.g. beach) by theme. The images would then be beach images with lots of blue sky. The same theme or category paired with a setting value for ‘low circadian impact’ would retrieve data files and produce image outputs of ‘beach sunset scenes.’ As another library image selection example, if the high red content images for alerting is desired, the processor might select data files from the library for images of red algae or reddish orange sunsets for output via the light source array.

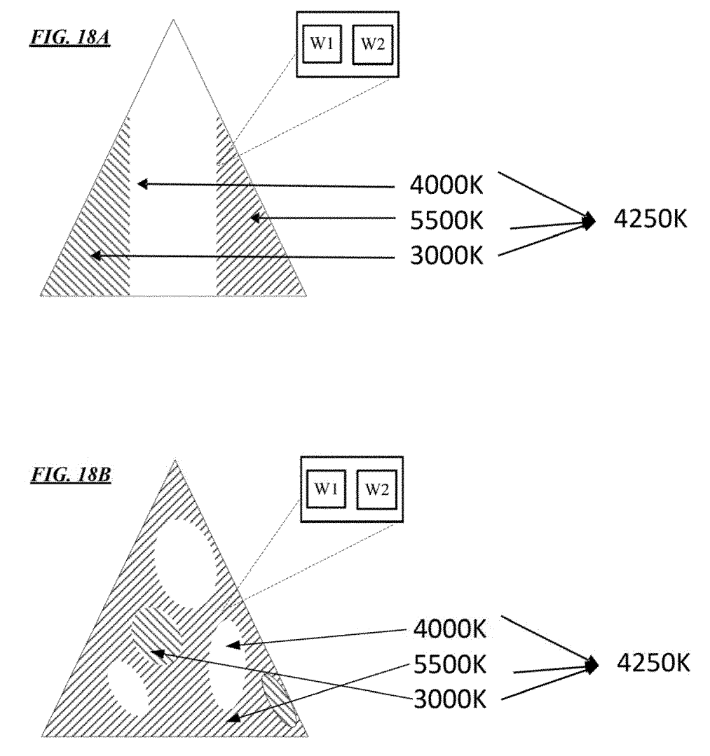

FIGS. 17A and 17B are a flow chart and associated normalized CCT distribution graph, respectively, for a two-channel white tuning example. For purposes of this example, the metric for the stimulus is melanopic lux, and a desired value for melanopic lux is obtained as a received input or as a value retrieved from storage in step S21.

The process flow branches at S23 based on the operational parameter that is to be optimized. Steps along one branch (‘power’) adjust light source operations to keep overall power consumption substantially constant (within an acceptable tolerance of a predetermined value for power consumption). Steps along the branch (‘CCT’) adjust light source operations to keep overall CCT of the overall light output from the source 110 within an acceptable tolerance of a predetermined value for the CCT of the output light. The overall process may optimize for power consumption, for CCT, or for both power consumption and CCT. The example, however, assumes selective optimize of just one of the two possible optimization parameters. The branch at S21 may be based on a user input, at the premises in real time or at a remote location during device/system configuration. Alternatively, the decision in the processing branch at S21 may be responsive to other control conditions, such as time of day, a sensed condition or the like.

Initially assume that processing branches at from step S21 to step S25. At step S25, a processor simultaneously solves for power consumption requirements and melanopic lux, based on the predetermined value for the power consumption parameter and the setting for melanopic lux. The processing in step S25 takes into account the spectral characteristics of the individual LEDs or the like used to implement the pixels of the emitter array of the particular light source 105. In this example, it is assumed that there are two types of white LEDs, such as 2700° K LEDs and 6500° K LEDs. FIG. 17B shows the normalized spectral power distributions for these two types of LEDs. Data 250 corresponding to the illustrated spectral power distributions is used in the processing to solve for the power consumption parameter and the setting for melanopic lux in step S25 (FIG. 17A).

For purposes of discussion, the first path based on maintaining power consumption assumes that power is the only parameter to be substantially maintained. Hence, other variables such as CCT and total lumen output variables dependent on the melanopic lux setting and the value for the optimized power parameter (see S27). The LEDs of the two white colors used as the individual emitters in the light source array are controlled (via the driver circuit) at step S27 of the process based on current values determined through steps S25, S27.

Returning to step S21, assume now that processing instead takes the branch for optimizing operation for a CCT parameter value. At S31, the processor uses the spectral data 250 to blend LED output values for the two types of white LEDs to maintain the predetermined value for the CCT parameter of source operation. Based on the melanopic lux values, the processor adjusts the blended values at S33 to dim the fixture output up or down and/or to vary color to meet the melanopic lux setting. Here, CCT is the optimized parameter and the drive currents for the two types of white LEDs for each pixel of the light source array are determined at S35 on the assumption that power consumption is a dependent variable and CCT is kept with an acceptable tolerance of the specified CCT parameter value. Again, the LEDs of the two white colors used as the individual emitters in the light source array are controlled (via the driver circuit) at step S27 of the process.

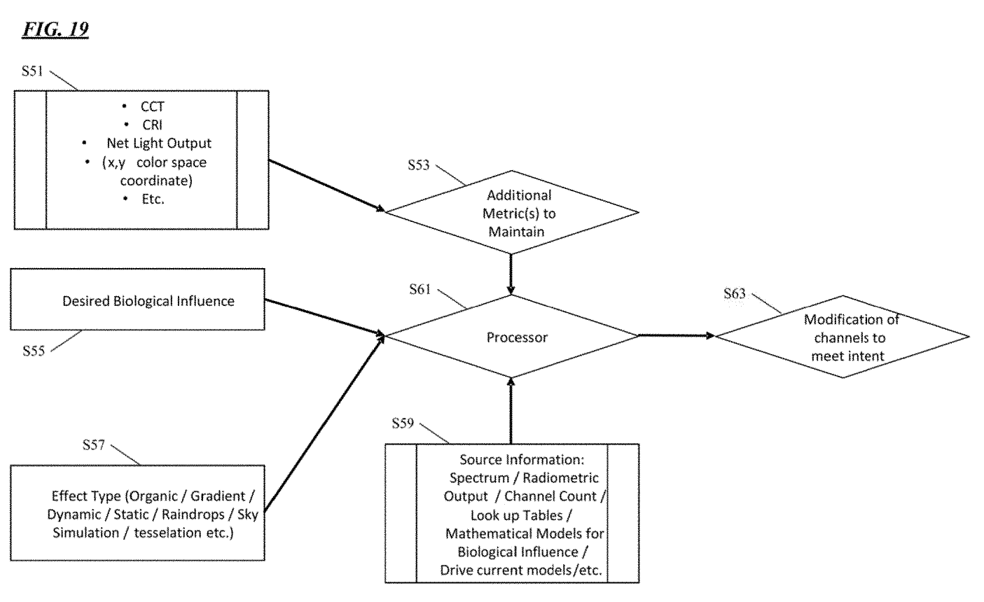

In a variant of image processing, an effect could be done by a pseudo-random algorithm. The algorithm for example, may obtain a style selection and generate organic-feeling shapes in a random way such as shapes analogous to blobs of ink spreading through water, and then balance the stimulus setting and operational parameter(s) algorithmically by changing the outputs of the channels of the emitters at the pixels of the array of the source 105 in a non-uniform way. FIGS. 18A and 18B depict two different states of a luminaire output providing the same coordinated color temperature (CCT) values for white light output, but which offer different visual effects. The different effects are selected, e.g. by a user; and the presentation of each selected effect may be manipulated by a pseudo-random processing algorithm to balance the stimulus setting and operational parameter(s). For discussion of this example, it is assumed that the light source array uses two-channel white emitters at the pixels of the array, such as the two different temperature white LEDs and the overall adjustment for melanopic lux and CCT as in part of the example of FIGS. 17A and 17B.

The pseudo-random algorithm may be static or dynamic. With this approach, a visual ‘effect’ is chosen and the lighting device or a processor communicating with the lighting device internally calculates what to portray on the visual light output of the light source 110. The visual effect may be implemented by choosing the ‘gradient effect’ and asking for a specific circadian output. FIGS. 18A and 18B show an example that blends the gradient in a way such that the biological effect is met, as well as the net CCT. FIG. 18A depicts a combined effect of blended gradients at transitions between areas and areas that are geometrically shaped (e.g. two triangles to the sides and a combination of a rectangle with a triangle in the middle). FIG. 18B depicts a combined effect of blended gradients at transitions between areas and blobs of different shapes overlaid on a background or filler area.

In the pseudo-random processing of FIGS. 18A and 18B, a control input parameter for this fixture would be a numeric of the user's choosing. Using the user input as a starting point, such as a seed for a pseudo-random number generator, the processor in or in communication with the lighting device 100 would generate data of an image with different areas of different shapes and color temperatures. In each of these drawings, each white area represents an area of white light output at 4000° K, each area with one type of cross-hatching (lines rising to the left) represents an area of white light output at 3000° K, and each area with the other type of cross-hatching (lines rising to the right) represents an area of white light output at 5500° K. Although not drawn to scale, the processing would design the total area of each type of white light output so as together to combine to provide a target spectral characteristic, which is 4250° K in the example. The shapes, locations, sizes and color temperatures are given by way of example only, and the pseudo-random algorithm could produce other shapes at other locations and sizes with different individual or combined color temperatures, for example, based on a different setting for melanopic lux and/or for a different target value for CCT or for another light source operational parameter.

Another variation of the pseudo-random algorithm approach could be to interlace or tessellate shapes which also gets fed into a circadian stimulus solving algorithm while balancing the above metrics.

The pseudo-random algorithm also may be beneficial in that it can create visual interest and inference through the CCT the time of day while giving the user the ability to at least somewhat customize the visual effect Also, the pseudo-random algorithm approach may be applied to other luminaire operation parameters or visual effects, such as localized color or hue shifts, instead of or in addition to blended CCT.

FIG. 19 is a simplified flow chart example of a general work flow for algorithmically determining intensity and spectral characteristics for the image pixels of the light output, which can be used to determine the drive signals for the emitters of the array. The workflow of FIG. 19 may be applied to the pseudo-random algorithm approach, as well as to other algorithmic and image processing techniques for balancing biological stimulus impact with one or more target luminaire operational parameters.

In the illustrated workflow, one or more target values for operational parameters are obtained in steps S51, S53. Depending on the particular control technique to be implemented, step S51 may involve a processor obtaining one or more parameter values related to overall light output of the luminaire 105, such as one or more of CCT, CRI, net light output, other metric of overall spectral characteristics (e.g. x,y color space coordinate), or the like. Step S53 may involve a processor obtaining one or more parameter values related to other aspects of luminaire operation to be maintained, such as power consumption.

In step S55, the processor obtains a metric value for a desired biological influence or stimulus impact, such as a setting value for: melanopic lux, circadian stimulus, circadian impact, alerting sensitivity of the human visual system, or the like. The stimulus metric, however, may be a metric for stimulus of another type of organisms, e.g. a metric for the impact of light on plant growth.

The parameter value(s) and stimulus metric setting obtained in steps S51 to S55 may be input by a designer or end user, selected based on a sensed condition or schedule, or determined by a processing algorithm.

For at least some techniques, such as the pseudo-random algorithm approach, the processor in step S57 obtains a selection of an effect type to be generated by the algorithm. For example, if the approach uses the pseudo-random algorithm technique of FIGS. 18A and 18B, step S57 may entail a user selection of an effect type, for example, one or more of: organic shapes, geometric shapes, gradient, dynamic, static, raindrops, sky simulation, tessellation, etc. The effect selection may be an automated function of the processor, e.g. based on schedule; or the selection may be responsive to a user input, as in the discussion of FIGS. 18A and 18B.

In step S59, the processor obtains source and computation related information as may be required for the particular control technique. The source Information may include information about the emitters of the array of the light source 110, such as spectrum, radiometric output, channel count, drive current models for the emitters, etc. In the example, S59 also provides the processor with look up tables or mathematical models for the particular type of biological influence.

In step S61, the processor implements one or more algorithms and/or image processing procedures based on the parameter values, settings, and/or visual effect selections obtained in steps S1 to S57 and on the characteristic information and modelling data obtained in step S59. In step S63, the processor outputs data specifying the emission modifications of the controllable channels of the emitters at the pixel points of the array of the particular light source 110. The outputs take the form of data to cause the driver circuit 155 to supply drive currents to the LEDs or the like of the emitter array to cause the light source 110 to generate light in a manner to meet the intent, that is to say to provide the currently desired biological stimulus, implement a selected visual effect and concurrently maintain one or more operational parameters of the luminaire 105 within tolerance of the respective target value.

The examples above rely on processing based in part on detailed spectral information about the capabilities of a particular light source 110. For LED based implementations of the emitter array of the light source 110, the techniques would utilize information on the LEDs used in the particular source array, such as spectrum, power consumption, and lumen output curves.

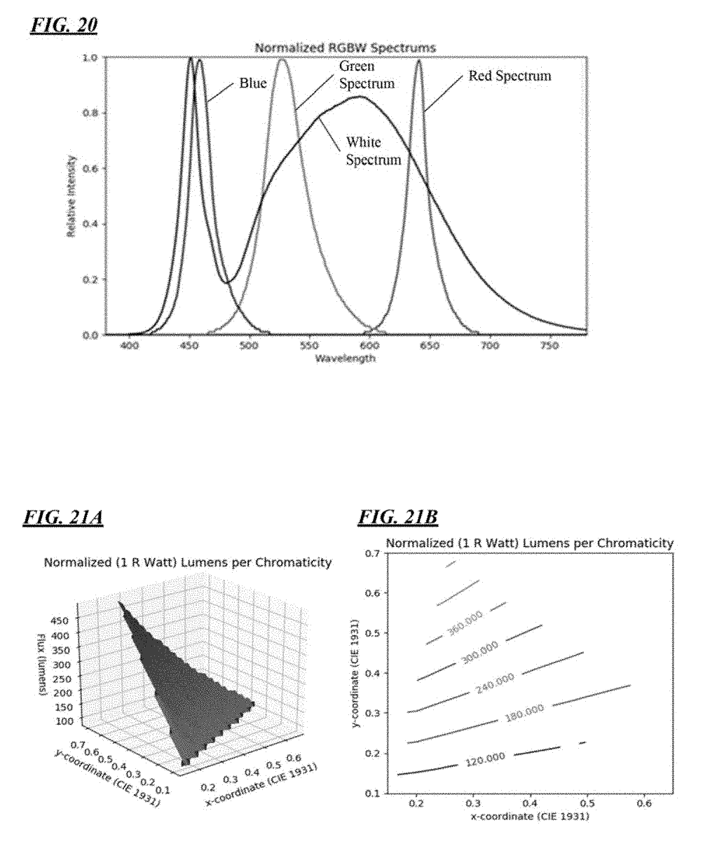

Across the chromaticity diagram there are specific surface functions which will be unique for any pixel spectrum combination such as RGB. For discussion purposes, the diagrams relate to RGB emitters, although a similar approach could be used for other channel combinations such as RGBW. FIG. 20 is a graph of normalized spectrum distributions for red (R), green (G), blue, (B) and white (W) LED type light emitters, useful in understanding the color gamut achievable with a light source array using such emitters, and the 2D color gamut triangular diagrams are well documented for different standards such as sRGB, Adobe RGB, etc.

The chromaticity surface functions represent how a specific chromaticity is generated and how much power, luminous efficacy, or melanopic efficacy the specific chromaticity has. The combined impact and operational parameter control as described here may be accomplished by utilizing these surface functions and applying an algorithm which utilizes real time calculation engines or pre-calculated information based on these surface function values across the available gamut.

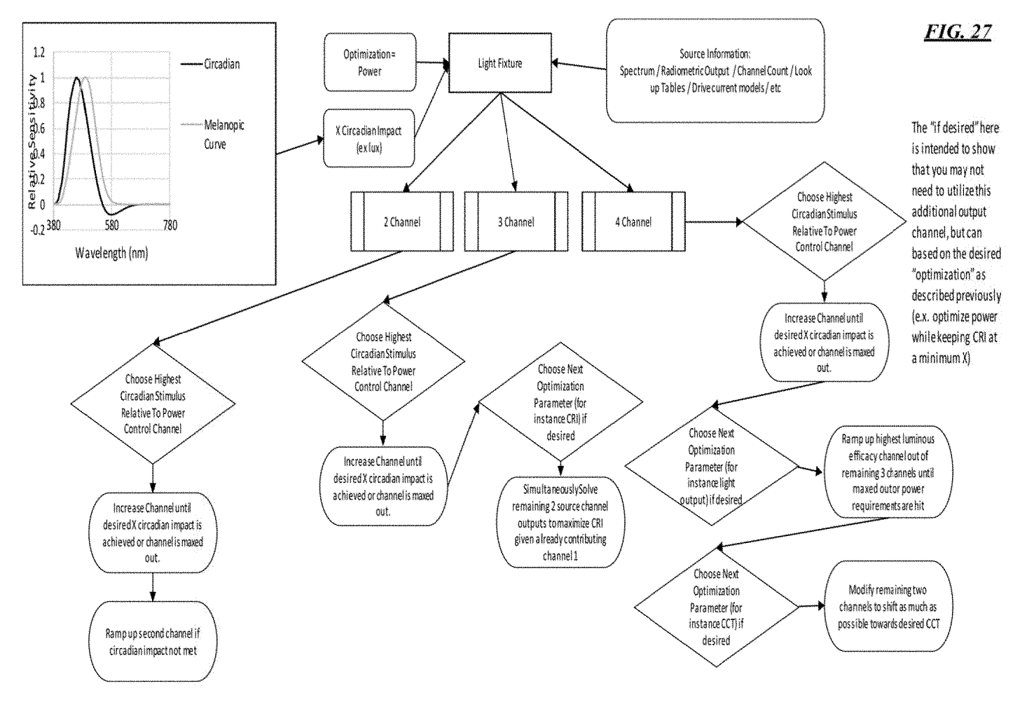

FIGS. 21A and 21 B are a gamut plot and graph regarding chromaticity, respectively, for Flux delivered for an RGB pixel with 1 radiometric watt of output. FIGS. 22A and 22 B are a gamut plot and graph regarding chromaticity, respectively, for flux delivered for an RGB pixel with 1 electric watt of input. FIGS. 23A and 23 B are a gamut plot and graph regarding chromaticity, respectively, for melanopic flux delivered for an RGB pixel with 1 radiometric watt of input. FIGS. 24A and 24 B are a gamut plot and graph regarding chromaticity, respectively, for melanopic flux delivered for an RGB pixel with 1 electric watt of input. FIGS. 25A and 25 B are a gamut plot and graph regarding chromaticity, respectively, for electric input power required to deliver 100 photopic lumens. FIGS. 26A and 26 B are a gamut plot and graph regarding chromaticity, respectively, for electric input power required to deliver 100 melanopic lumens. These gamuts and graphs were generated based on an RGB LED type pixel emitter, for simplicity, but can also be generated with any other style of pixel emitter. The normalized RGB spectrums used for this part of the discussion are shown in FIG. 20. Actual values for a LED type RGB pixel emitter were utilized to generate the information in FIGS. 20-26B, including electric-radiometric efficiencies, ignoring effects of temperature and non-linear flux output with current.

The illustrated chromaticity surface functions show that there are specific areas within a chromaticity gamut (defined by the multiple spectrums within a pixel), which are better suited for delivering photopic flux (like the greenish area of the chromaticity diagram) or melanopic lux (like the blueish area of the chromaticity diagram) which have their root in the sensitivity functions. These chromaticity surface functions can be applied to calculate LED drive values, by utilizing matrix multiplication to simultaneously solve for certain desired aspects such as melanopic lux and CRI within the limits of the light source.

Based on the chromaticity information examples in the drawings, an example application might use several points on the surface or contour plots which move around in a non-uniform way based on what a lighting device or a higher level system data-processing component is attempting to balance in the operation of the luminaire 105. Consider, for example three points on the melanopic flux per electric input watt on the gamut represented by FIG. 24A. Any three points on the chromaticity surface will add up to a net of 3 electric Watts, but melanopic flux could be increased or decreased (and effect other factors in a balanced way) by adjusting the chromaticity outputs (and maintaining electric input power) of the three points (and corresponding outputs of some number of pixels of the emitter array).

The parameters being modified by changing the image could be for any biological benefits (not limited to Circadian): e.g. circadian impact, absence of circadian impact, alerting, grow lights (e.g. for indoor spaces with grow walls), or any combination of these. The goal may or may not be to also produce usable general illumination.

A variety of additional or alternative operational parameters were mentioned in the earlier examples; and additional or alternative impact metrics were mentioned earlier. While these other parameters or metric settings are not shown examples of FIGS. 21A to 26B, a similarly methodology can apply and generate similar chromaticity data, using appropriate functions based in spectral calculations.

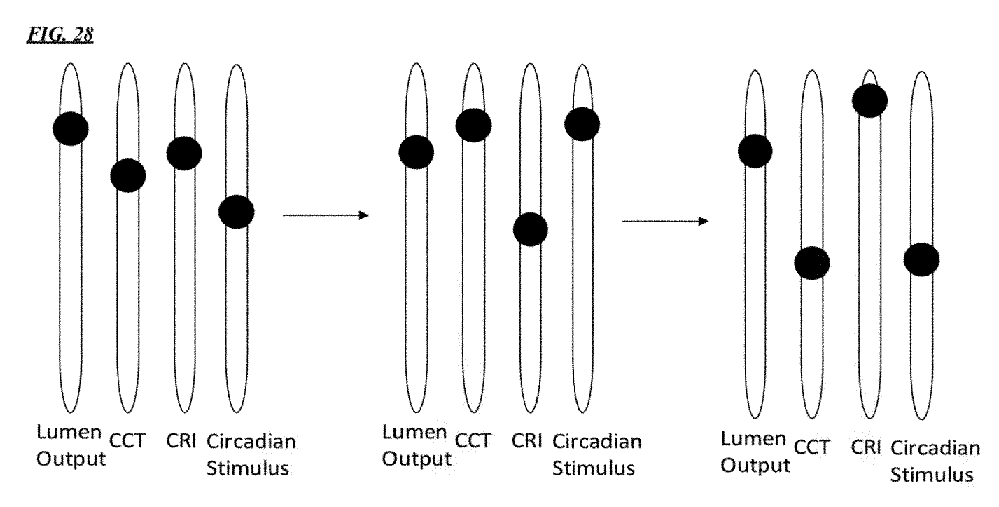

It also may be desirable in some circumstances to control the channels of the light source 110 in such a way that one or more operational parameters are optimized, rather than simply maintained, for example, to maximize net light output, maximize color rendition or specific subset of spectrum to make certain things look good (for instance fruit), or minimize power consumption. Optimization here could be somewhat subjective, such as “maintain CCT as close as possible without power consumption going over X watts.” FIG. 27 is a flow chart of a process for factor optimization. FIG. 28 graphically illustrates several lighting parameters and related stimulus impact values, for several different outcomes of the factor optimization process of FIG. 27. Based on “channel count” where channel count is quantity and type of individual light spectrum output (for instance, W1W2=2 channels, RGB=3 channels, and RGBW=4 channels), there is a theoretical limit on the optimization parameters (n−1) and depending on the order and choice of optimization. Optimization parameters in the example of FIGS. 27 and 28 may include power consumption, net light output Color rendition or net CCT output. This optimization process utilizes metamers to keep a visual appearance on a luminaire (e.g. maintain a color space XY coordinate output).

The design of the luminaire and channels may be such that not an entire metric is met by one channel online (making the optimization algorithms more important). For instance a desired biological effect may not be achievable by running one control channel at maximum, a minimum of two must be used, but they can be driven in different ways depending on desired optimization parameter.

The steps of the example optimization process flow should be fairly apparent from the texts included in the blocks of the flow chart in FIG. 27.

FIG. 28 shows optimization results, like from iterations of the process of FIG. 27, in a more conceptual manner. In a 3+ channel system, the complexity of optimization becomes exponential. Because of the increased complexity, it may be advantageous to use a pre-calculated relational equations, based on physical outputs of the light fixture to affect biological systems, to represent the different metrics of input and output and the relationship between those metrics. FIG. 28 depicts adjustment of the stimulus setting (circadian in in the illustrated example) and operational parameters as if represented on a user-interface display showing a set of sliders (or other set of user-interface controls), for example, based on such pre-calculated relational equations. Each slider could be represented in a numerical scale or in another format such as relative scale. Effectively, when one slider or control is modified, the others adjust in real time to show the relation to each other. For instance, the sliders of FIG. 28 could have an initial state shown on the left, and when modified moved in real time to show the metric change to the center state, and the further changes from the center state to the right state.

From the discussions above, it will be apparent that many of the processing and control functions of lighting and providing associated human stimulus impact may be implemented in part by utilizing software or firmware, for example, using executable instructions of a program and/or data for manipulation by a processor. Also, the processing functions may be implemented in a lighting device 100 (e.g. as shown in FIG. 9). Alternatively, some or all of the processing functions may be implemented via communication with general purpose computers or other general purpose user terminal devices (e.g. FIG. 11), although special purpose devices may be used. FIGS. 29 to 31 provide functional block diagram illustrations of exemplary general purpose hardware platforms that may be used in a system like 200 of FIG. 11.

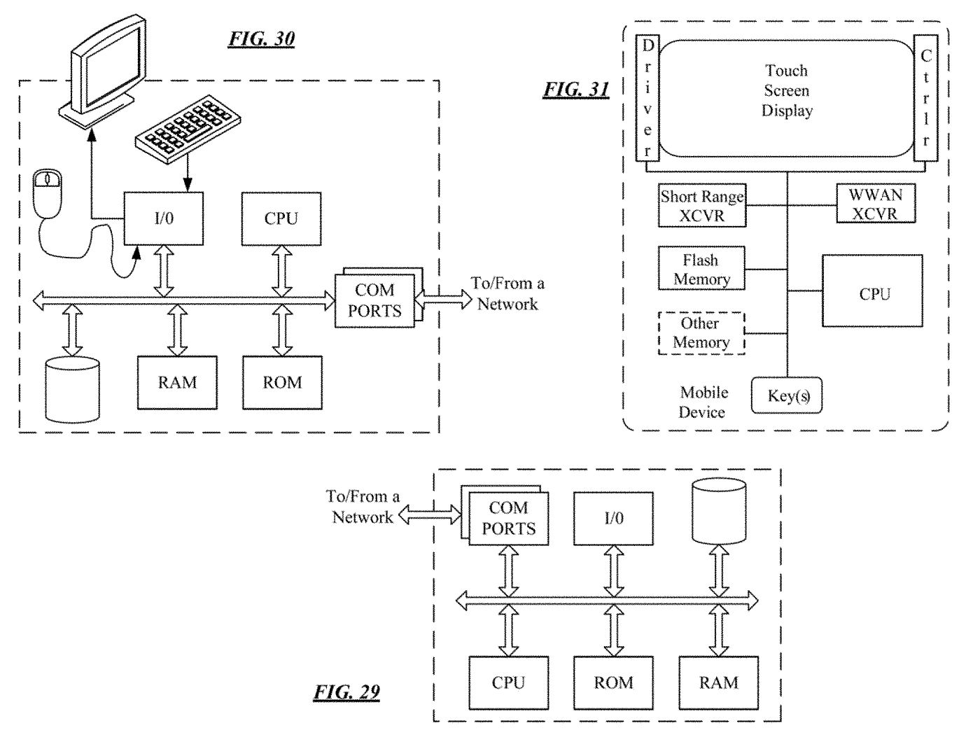

FIG. 29 illustrates a network or host computer platform, as may typically be used to generate, send and/or receive images, updated image, lighting control settings or commands, configuration files, programming or formulas for algorithms, algorithm initial values or coefficients, schedules, or the like, as well as to access networks and devices external to the lighting device 100, for example, to implement the server 229 and/or the database 231 of in the system of FIG. 11. FIG. 30 depicts a computer with user interface communication elements, such as terminal 227 as shown in FIG. 11, although the computer of FIG. 30 may also act as a server if appropriately programmed. The block diagram of a hardware platform of FIG. 31 represents an example of a mobile device, such as a tablet computer, smartphone or the like with a network interface to a wireless link, which may alternatively serve as a user terminal device for providing a user communication with a lighting device, such as device 100, or with a server 229. It is believed that those skilled in the art are familiar with the structure, programming and general operation of such computer equipment and as a result the drawings should be self-explanatory.

A server (see e.g. FIG. 29), for example, includes a data communication interface for packet data communication via the particular type of available network. The server also includes a central processing unit (CPU), in the form of one or more processors, for executing program instructions. The server platform typically includes an internal communication bus, program storage and data storage for various data files to be processed and/or communicated by the server, although the server often receives programming and data via network communications. In general, the hardware elements, operating systems and programming languages of such servers may be conventional in nature. Of course, the server functions may be implemented in a distributed fashion on a number of similar platforms, to distribute the processing load. A server, such as that shown in FIG. 29, may be accessible or have access to a lighting device 100 via the communication interfaces 190 or the wireless transceiver 180 of the lighting device 100. For example, the server may respond to a user request for an image and/or a configuration information file to send the requested information to a communication interface 190 of the lighting device 100 for installation in memory 170 for future use. The information of a configuration information file may be used to configure a lighting device, such as lighting device 100, to set light output parameters for general illumination purposes such as light intensity or light spectral characteristic, other operational parameters such as power consumption, various parameter tolerances, and/or to set control variation of vary intensity or color characteristic or spectral power distribution of emitted light so as to promote the desired human stimulus impact, in any of the various ways described herein.

A computer type user terminal device, such as a desktop or laptop type personal computer (PC), similarly includes a data communication interface CPU, main memory (such as a random access memory (RAM)) and one or more disc drives or other mass storage devices for storing user data and the various executable programs (see FIG. 30). A mobile device (see FIG. 31) type user terminal may include similar elements, but will typically use smaller components that also require less power, to facilitate implementation in a portable form factor. The example of FIG. 31 includes a wireless wide area network (WWAN) transceiver (XCVR) such as a 3G to 5G cellular network transceiver as well as a short range wireless transceiver such as a Bluetooth, WiFi, and/or ultra-wide band transceiver for wireless local area network (WLAN) communication. The computer hardware platform of FIG. 30 and the terminal computer platform of FIG. 31 are shown by way of example as using a RAM type main memory and a hard disk drive for mass storage of data and programming, whereas the mobile device of FIG. 31 includes a flash memory and may include other miniature memory devices. It may be noted, however, that more modern computer architectures, particularly for portable usage, are equipped with semiconductor memory only.

The various types of user terminal devices will also include various user input and output elements. A computer, for example, may include a keyboard and a cursor control/selection device such as a mouse, trackball, joystick or touchpad; and a display for visual outputs (see FIG. 30). The mobile device example in FIG. 31 uses a touchscreen type display, where the display is controlled by a display driver, and user touching of the screen is detected by a touch sense controller (Ctrlr). In general, the hardware elements, operating systems and programming languages of such computer and/or mobile user terminal devices also are conventional in nature.

The user device of FIG. 30 and the mobile device of FIG. 31 may also interact with the lighting device 100 in order to enhance the user experience or to initially configure the lighting device 100 and/or for operation in a system 200, to provide lighting and associated variation to promote a human stimulus impact as described herein.

For example, third party applications stored as programs on such terminal equipment may correspond to programming 127 at the device 100, to allow the user to manipulate controllable functions of a lighting device 100, such as image display and general illumination lighting settings or even settings or schedules related to light variations to provide a desired human stimulus impact.

As also outlined above, aspects of the techniques for operation of a lighting device 100 with the luminaire 105 and any system interaction therewith, may involve some programming, e.g. programming of the lighting device 100 or any server or terminal device in communication with the lighting device. For example, the mobile device of FIG. 31 or the user device of FIG. 30 may interact with a server, such as the server of FIG. 29, to obtain configuration information that may be delivered to a lighting device 100. Subsequently, the mobile device of FIG. 31 and/or the user device of FIG. 30 may execute programming that permits the respective devices to interact with the lighting device 100 to provide control commands such as the ON/OFF command, an image selection or a performance command, such as dimming. The processor 123 of the lighting device 100 in turn runs its programming 172 to control the light source 110 of the luminaire 105, e.g. in accordance with one or more received images and/or in accordance with received settings.

Program or data aspects of the technology discussed above therefore may be thought of as “products” or “articles of manufacture” typically in the form of executable programming code (software or firmware) or data that is carried on or embodied in a type of machine readable medium. At least one medium, for example, may carry image data and an illumination light setting. Programming or control data also is embodied in the at least one medium. This programming or control data is configured to implement control of operation of a light source 110 of the luminaire 105 to provide lighting with a variation to promote a particular human stimulus impact in one or more of the ways described above.

“Storage” type media include any or all of the tangible memory of lighting devices, computers, user terminal devices, intelligent standalone sensors, processors or the like, or associated modules thereof, such as various volatile or non-volatile semiconductor memories, tape drives, disk drives and the like, which non-transitory devices may provide storage at any time for executable software or firmware programming and/or any relevant data or information. All or portions of the programming and/or configuration data may at times be communicated through the Internet or various other telecommunication networks. Such communications, for example, may enable loading of the data or programming from one computer or processor into another, for example, from a management server or host computer of a lighting system service provider into any of the lighting devices 100, or other non-lighting-system devices, etc. Thus, another type of media that may bear the programming or data elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible or “storage” media, terms such as computer or machine “readable medium” refer to any medium that participates in providing instructions to a processor for execution.

The image data, light setting data, and programming or data for lighting with human stimulus impact may be embodied in at least one machine readable medium, one or more of which may be non-transitory. For example, if downloaded to a lighting device 100, the image data, light setting data, and programming or data could be stored in a hardware device that serves as the memory/storage 170 of the host processing system 160. The memory/storage 170 is an example of a non-transitory type of media. By way of another example, at times, executable operational programming, including programming and/or data for the interference control strategy, may reside in the memory/storage 170, while actual image data and/or associated general illumination light setting data is transmitted in real time via a network medium. Interference control data may reside in memory 125 or be streamed over the network medium. In these later examples, the signal(s) on the network would be transitory in nature. However, the buffer memory and any memory or registers internal to the processor memory, or any hardware storage device used by the server to maintain the database or prepare selected data for transmission over the network would be additional examples of non-transitory media.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that comprises or includes a list of elements or steps does not include only those elements or steps but may include other elements or steps not expressly listed or inherent to such process, method, article, or apparatus. An element preceded by “a” or “an” does not, without further constraints, preclude the existence of additional identical elements in the process, method, article, or apparatus that comprises the element.

Unless otherwise stated, any and all measurements, values, ratings, positions, magnitudes, sizes, and other specifications that are set forth in this specification, including in the claims that follow, are approximate, not exact. Such amounts are intended to have a reasonable range that is consistent with the functions to which they relate and with what is customary in the art to which they pertain. For example, unless expressly stated otherwise, a parameter value or the like may vary by as much as ±10% from the stated amount.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus the following claims are hereby incorporated into the Detailed Description, with each claim standing on its own as a separately claimed subject matter.

While the foregoing has described what are considered to be the best mode and/or other examples, it is understood that various modifications may be made therein and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. A lighting device, comprising:
a luminaire having a light source, the light source including:
a plurality of independently controllable light emitters configured to emit light, wherein:
each respective light emitter is configured to be controlled to vary intensity or a color characteristic of light emitted from the respective light emitter, and
the plurality of individually controllable light emitters together are configured to be able to emit light in a manner meeting a predetermined level of an operational parameter of the light source; and
a controller coupled to control the plurality of controllable light emitters, the controller being configured to:
obtain a setting corresponding to a desired human stimulus impact to be provided by the light emitted by the plurality of controllable light emitters;
based on the setting, control at least one of the light emitters to vary intensity or color characteristic or spectral power distribution of light emitted by the at least one light emitter to promote the desired human stimulus impact; and
during the variation of the intensity or color characteristic or spectral power distribution of light emitted by the at least one light emitter, control operation of the emitters of the light source to maintain operation at least within a tolerance of the predetermined level of the parameter of operation the light source.

2. The lighting device of claim 1, wherein the plurality of independently controllable light emitters comprise an array of controllable pixel emitters, each of the controllable pixel emitters including at least two individual emitters controllable to emit light having different spectral characteristics.

3. The lighting device of claim 2, wherein the individual emitters of each of the controllable pixel emitters comprise a plurality of light emitting diodes which emit light of different color characteristics.

4. The lighting device of claim 1, wherein the parameter of operation is a parameter selected from the group consisting of color rendering index, correlated color temperature, image resolution, illumination intensity and power consumption.

5. The lighting device of claim 1, wherein the desired human stimulus impact is a stimulus impact to be provided by the light emitted by the plurality of controllable light emitters selected from the group consisting of circadian stimulus, circadian impact, melanopic lux, and alerting sensitivity of the human visual system.

6. The lighting device of claim 1, wherein the controller comprises
a processor:
a memory coupled to the processor; and
data of an image stored in the memory,
wherein the processor is configured to:
receive the data of the image from the memory; and
control the plurality of controllable light emitters, based on the received data, to emit light to present the image.

7. The lighting device of claim 6, wherein the processor is further configured to:
at a later time, process the data of the image to produce processed image data; and
control the plurality of controllable light emitters, based on the processed image data.

8. The lighting device of claim 1, wherein the controller comprises:
a processor;
a memory coupled to the processor; and
programming for an algorithm, stored in the memory, wherein execution of the programming causes the processor to control the plurality of controllable light emitters to emit light to generate one or more patterns of features.

9. The lighting device of claim 8, wherein the one or more patterns are time-varying.

10. The lighting device of claim 8, wherein the one or more patterns are pseudo-random patterns.

11. The lighting device of claim 1, wherein the controller comprises:
a processor;
a memory coupled to the processor; and
programming for an algorithm, stored in the memory, wherein execution of the programming causes the processor to:
determine the desired human stimulus impact based on the algorithm;
determine the setting corresponding to the desired human stimulus impact according to the algorithm stored in the memory; and
control the plurality of controllable based on the determined setting.

12. The lighting device of claim 11, wherein the processor is further configured to obtain a value of an external variable and determine the setting corresponding to the desired human stimulus impact from application of the algorithm to the value of the external variable.

13. The lighting device of claim 12, wherein the external variable is a variable selected from the group consisting of time of day, ambient light, and ambient noise.

14. A lighting device, comprising:
a luminaire having a light source,
the light source including an array of controllable light emitters configured to generate light output representing an image,
wherein each respective light emitter is configured to be controlled to vary intensity and a color characteristic of light emitted from the respective light emitter;
a processor coupled to control the controllable light emitters; and
a memory coupled to the processor, the memory storing data of each of a plurality of images,
wherein the processor is configured to:
obtain: a setting corresponding to a desired human stimulus impact to be promoted by light emitted by the controllable light emitters, and a value specifying a predetermined level of a parameter of operation of the light source;
select one of the plurality of images which will promote the desired human stimulus impact;
based on the data of the selected image, control the controllable light emitters to generate light output representing the selected image; and
concurrently control the controllable light emitters to of the source to maintain operation at least within a tolerance of the predetermined level of the parameter of operation the light source.

15. The lighting device of claim 14, wherein the independently controllable light emitters comprise an array of controllable pixel emitters, each of the controllable pixel emitters including at least two individual emitters controllable to emit light having different spectral characteristics.

16. The lighting device of claim 15, wherein the individual emitters of each of the controllable pixel emitters comprise a plurality of light emitting diodes which emit light of different color characteristics.

17. The lighting device of claim 14, wherein the parameter of operation is a parameter selected from the group consisting of color rendering index, correlated color temperature, image resolution, illumination intensity and power consumption.

18. The lighting device of claim 14, wherein the desired human stimulus impact is a stimulus impact to be provided by the light emitted by the controllable light emitters selected from the group consisting of circadian stimulus, circadian impact, melanopic lux and alerting sensitivity of the human visual system.

19. The lighting device of claim 14, wherein:
the memory further stores an algorithm for identifying the desired human stimulus impact, and
the processor is further configured to identify the desired human stimulus impact according to the algorithm stored in the memory.

20. The lighting device of claim 19, wherein the processor is further configured to obtain a value of an external variable and identify the desired human stimulus impact from application of the algorithm to the value of the external variable.

21. The lighting device of claim 20, wherein the external variable is a variable selected from the group consisting of time of day, ambient light, and ambient noise.

22. The lighting device of claim 14, wherein:
the memory stores the data of each of the plurality of images in association with one of a plurality of themes,
the processor is further configured to obtain a desired image theme of the plurality of themes; and
the processor is further configured to select the one of the plurality of images from among images associated with the desired image theme.

23. The lighting device of claim 14, wherein the processor is further configured to, after a predetermined period of time:
select a new one of the plurality of images which will promote a desired human stimulus impact;
based on the data of the newly selected image, control the controllable light emitters to emit light to generate light output representing the newly selected image; and
concurrently control the controllable light emitters to maintain operation of the light source at least within the tolerance of the predetermined level of the parameter.

24. The lighting device of claim 14, wherein the processor is further configured to, after a predetermined period of time:
process the data of the selected image to produce processed image data; and
control the controllable light emitters, based on the processed image data; and
concurrently control the controllable light emitters to maintain operation of the light source at least within the tolerance of the predetermined level of the parameter.
